(19) United States
(12) Patent Application Publication
(10) Pub. No.: US 2005/0276767 A1
(43) Pub. Date: Dec. 15, 2005

(54) Composition containing an oily phase and a naphthopyran dye, cosmetic treatment processes

(75) Inventors: Xavier Blin, Paris (FR); Jean-Christophe Simon, Alsbach-Hahnlein (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/687,581

(22) Filed: Oct. 20, 2003

Related U.S. Application Data
(60) Provisional application No. 60/426,411, filed on Nov. 15, 2002.
(60) Provisional application No. 60/426,376, filed on Nov. 15, 2002.

(30) Foreign Application Priority Data
Oct. 18, 2002 (FR) 0213003
Oct. 18, 2002 (FR) 0213004

(51) Int. Cl. A61K 7/021

(52) U.S. Cl. 424/63

(57) ABSTRACT

The present patent invention relates to a cosmetic composition containing, preferably in a cosmetically acceptable medium, at least one oily phase and at least one dye selected from the group consisting of 3H-naphtho[2,1-b]pyrans and 2H-naphtho[1,2-b]pyrans. Cosmetic treatment process, and especially a makeup and/or temporary coloring process, for a support selected from the group consisting of mucous membranes, semi-mucous membranes, the skin and/or the integuments, in which a cosmetic composition as defined above is applied to the support.

### REFERENCE TO PRIOR APPLICATIONS

This application claims priority to U.S. provisional application 60/426,376 and 60/426,411, both filed Nov. 15, 2002, and to French patent applications 0213003 and 0213004 both filed Oct. 18, 2002, all incorporated herein by reference.

### FIELD OF THE INVENTION

The present invention relates to novel compositions and especially novel makeup compositions, comprising organic and especially photochromic dyes.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Makeup compositions such as free or compact powders, foundations, makeup rouges, eyeshadows or lipsticks generally consist of a suitable vehicle and of one or more coloring agents intended to give the compositions a certain color before and/or after they are applied to the skin, mucous membranes, semi-mucous membranes and/or the integuments such as the nails or the hair.

To create colors, a fairly limited range of coloring agents is used at the present time, especially comprising lakes, mineral pigments, organic pigments and nacreous pigments. Lakes make it possible to obtain vivid colors, but most of them are unstable to light, temperature or pH. Some also have the drawback of making an unsightly stain on the skin after application, due to running of the dye. Mineral pigments, in particular mineral oxides, are, in contrast, very stable, but give rather bland and pale colors. As for nacreous pigments, they produce varied but not very intense colors, which lead to iridescent effects, but these are usually quite weak.

In the field of temporary or short-term dyeing of the hair, which gives rise to a mild change in the natural color of the hair which holds from one shampoo wash to the next and which serves to enhance or correct a shade already obtained, it has already been proposed to dye the hair with common pigments to give it a temporary glint, but the shades obtained by this coloration remain fairly bland, too uniform and quite lacking in interest. Such a coloration may especially be likened to a hair “makeup”.

It has also recently been proposed to use photochromic compounds in makeup or hair compositions, so as to obtain pleasant, interesting and variable changes in the “rendered color” of the skin and/or hair makeup results.

Photochromic compounds are compounds that have the property of changing color when they are irradiated with a light source, and then of regaining their initial color, or a similar color, when the irradiation is stopped. Such compounds especially find a particularly advantageous application in cosmetic compositions, in particular in makeup compositions such as foundations, makeup rouges, eyeshadows and lipsticks.

Specifically, it has been found that the “makeup result” of made-up skin is different depending on whether the wearer is under natural light or artificial lighting. Thus, a makeup applied under artificial lighting will appear lighter in color under natural light. Conversely, a makeup applied out of doors will appear darker in an artificially lit place. To overcome this problem, cosmetic compositions comprising particular mineral photochromic compounds, selected from the group consisting of metal oxides, hydrates thereof and complexes thereof, have been proposed, for example by patent EP 359 909. In particular, the document mentions the use of titanium oxide treated so as to render it photochromic, in makeup compositions such as powders and foundations. However, it has been found that these mineral photochromic compounds, although producing a makeup that appears to remain a constant color irrespective of the lighting, do not, however, produce a genuine change in the color of the makeup, or, in other words, a genuine change in the “makeup result”.

Moreover, it has also been found that, after stopping the light irradiation, the color of the makeup did not always return in an acceptable manner to its initial color, and in particular that it did not completely return to a color identical to the initial color and/or not quickly enough.

It has also been proposed to use organic photochromic compounds, such as compounds of the spiropyran or naphthoxazine family. These photochromic compounds are particularly advantageous since they produce a rapid change in the coloration of the support onto which they are applied, when the support is exposed to UV, for example, with a rapid return to the initial color when the UV exposure ceases. Mention may thus be made of patent FR 1 604 929, which describes cosmetic compositions, especially hair compositions in aerosol form, which contain phototropic compounds such as nitrobenzylpyridines, thiosemicarbazones or spiropyran derivatives. After spraying these compositions onto the hair and exposing it to sunlight, a violet-blue coloration is obtained, which becomes pale yellow in the dark.

Also, EP 970 689 discloses a cosmetic makeup product comprising a first and a second composition packaged separately, the first composition containing a photochromic coloring agent capable of producing at least one color in the presence of UV, and the second composition containing at least one UV-screening agent. However, the dynamics of these compositions are poor: in particular, the return to the initial state is slow. Also, patent application WO 02/078665 discloses a cosmetic composition comprising UV-absorbing liquid dyestuffs, of naphthopyran or naphthoxazine type, which are encapsulated in a capsule based on acrylate copolymers, and which can be used for the purpose of absorbing UV. However, there is no mention at all of any photochromic property for these compositions or dyestuffs.

### DETAILED DESCRIPTION OF THE PREFERRED EMBODIMENTS

The present invention overcomes some of the drawbacks of the prior art, by means of novel, preferably cosmetic, compositions comprising particular organic dyes, in combination with a particular oily phase, which may be, but is not necessarily, at least partially silicone-based.

It is known that, among the oils that may be used in cosmetics, silicone oils provide particular properties such as a certain amount of slipperiness and good film formation. In particular, on applying them to the skin or the lips, they lead to the formation of a deposit with satisfactory uniformity, softness and sheen. Moreover, their good wetting property gives them resistance to attack, especially water resistance, which contributes towards the good staying power of the composition.

It may thus be particularly advantageous to have available a cosmetic composition comprising both such silicone oils and photochromic dyes as defined below.

However, the inventors have found that it was not possible to formulate a cosmetic composition comprising the combination of the photochromic dyes and silicone oils usually used, especially since the dyes were not dissolvable in these silicone oils.

The inventors have, to theri credit, discovered a means for allowing the dissolution, and thus the formulation, of these photochromic dyes in silicone oils.

A first subject of the present invention is a composition comprising, in a cosmetically acceptable medium, at least one oily and especially silicone-based phase and at least one dye as defined below. These compositions and all those described in other places herein are preferably “cosmetic compositions,” meaning that they are in a form and of a type acceptable for cosmetic use.

Specifically, it has been found, surprisingly, that dyes and especially photochromic dyes are most particularly soluble in phenylsilicone oils; it thus becomes possible to formulate them either, for example, by using only phenylsilicone oils in the oily phase of the composition, or by predissolving them in the phenylsilicone oils and then adding to the solution the other oils that it is desired to use, which may or may not be silicone-based. In this second case, the phenylsilicone oils are thus used as “solubilizers” or “compatibilizers” for the dyes.

In addition, it has been found, surprisingly, that, depending on the polarity of the oil or of the mixture of oils forming the oily phase of the cosmetic composition, it is possible to modify the color of the initial composition, before irradiation.

Thus, when the oily phase is polar or relatively polar, it has in the presence of the photochromic dyes according to the invention a coloration that can range, for example, from pale pink to violet passing through orange, depending of course on the chemical nature of the dye, the amount thereof and also, especially, the polarity and the chemical nature of the oily phase.

When the oily phase is relatively apolar or weakly polar, it has been found that its color is not perturbed or perturbed only very little by the presence of the photochromic dyes before irradiation; the oily phase remains virtually colorless.

The photochromic dyes according to the invention are thus also solvatochromic, i.e. they change color as a function of the nature (polarity) of the solvent they are in.

This especially makes it possible, for example, to obtain a photochromic cosmetic product that is completely colorless without UV excitation, and then a split effect (colorless/colored) after irradiation. Furthermore, if the composition is otherwise pigmented, the absence of color provided by the photochromic dye according to the invention makes it possible to leave the initial color undisturbed.

A photochromic cosmetic product that is mildly colored without UV may also be obtained, which can make it possible to visualize its deposit and to facilitate its application before excitation and intensification of the color.

By virtue of the dyes, especially photochromic and solvatochromic dyes, according to the invention, it is possible not only to modify the color imparted by a cosmetic composition comprising the dyes, by irradiation (photochromism), but also to modify the color of the initial composition, before irradiation.

Another subject of the invention is a composition comprising, in a cosmetically acceptable medium, at least one oily phase comprising, for example, a polar and/or apolar oil and at least one dye as defined below.

The (cosmetic) compositions according to the invention advantageously make it possible to obtain a makeup that can change color quickly depending on the nature and/or intensity of the light.

The use of the dyes according to the invention also makes it possible to obtain a makeup of sharp and pure color, with a “makeup result” that appears more intense.

The color change obtained using these dyes is rapid; it is also reversible and makes it possible to return to the initial color after stopping the irradiation from a light source, this return also occurring rapidly.

Moreover, it is thus possible to use, in the cosmetic composition according to the invention, a smaller amount of photochromic dyes than that generally used in the prior art, for example from about 0.01% to 2% by weight, while at the same time obtaining a comparable makeup effect and a comparable transparency.

In addition, the good cosmetic properties of the compositions according to the invention are maintained. Furthermore, although of similar chemical structure, the photochromic dyes according to the invention present a wide variety of colors, which may range from red to blue passing through grey, with more or less dark variations. This especially allows easier formulation of the dyes, since it is not necessary to adapt the composition for each change in shade.

The organic dyes according to the invention are naphthopyrans, and preferably are 3H-naphtho[2,1-b]pyrans, which may be represented by formula (I), or 2H-naphtho[1,2-b]pyrans which may be represented by formula (II): 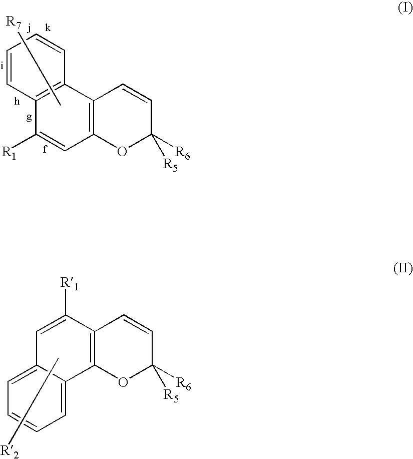
in which:
R1 represents:
(i) a hydrogen atom;
(ii) a linear, branched or cyclic, saturated or unsaturated hydrocarbon-based group containing from 1 to 30, preferably 1-18 and better still 1 to 12, or even 1 to 6, carbon atoms, optionally comprising 1 to 5 hetero atoms selected from the group consisting of N, O, S, Si and P, and/or optionally halogenated or perhalogenated, especially with F, Br and/or Cl;
(iii) a hydrocarbon-based ring formed with one of the bonds “f” or “gh” and the radical R7; or
(iv) a group selected from the group consisting of —COOR4, —C(O)NR2R3, —NR2R3, —OR4 and —SR4, in which:
   R2 and R3 either represent, independently of each other, a linear, branched or cyclic, saturated or unsaturated hydrocarbon-based group containing 1 to 20, preferably 1-12 and better still 1 to 6 carbon atoms, optionally comprising 1 to 5 hetero atoms selected from the group consisting of N, O, S, Si and P, or, taken together with the nitrogen atom to which they are attached, form a saturated or unsaturated hydrocarbon-based heterocycle containing 3-10 and preferably 4-6 carbon atoms and optionally 1-5 other hetero atoms selected from the group consisting of N, O, S, Si and P, the ring optionally being substituted with at least one linear, branched or cyclic, saturated or unsaturated hydrocarbon-based radical containing 1 to 20, preferably 1-15 and better still 1 to 6 carbon atoms, optionally comprising 1 to 5 hetero atoms selected from the group consisting of N, O, S, Si and P;
   R4 represents a linear, branched or cyclic, saturated or unsaturated hydrocarbon-based group containing 1 to 20, preferably 1-12 and better still 1 to 6 carbon atoms, which is optionally halogenated or perhalogenated especially with F, Br and/or Cl, and/or optionally comprising 1 to 5 hetero atoms selected from the group consisting of N, O, S, Si and P;
R5 and R6 represent, independently of each other, a group selected from the group consisting of:
(i) the saturated cyclic aminoaryl groups of formula (IIa) or (IIb): 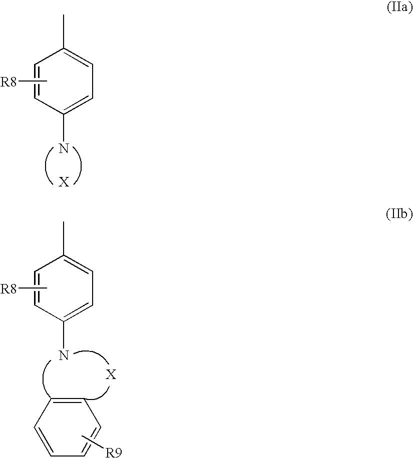
in which the ring comprising N and X is a saturated ring containing in total 3 to 30 atoms, preferably 4-10 and better still 5, 6 or 7 atoms, including the nitrogen, the remainder being carbon atoms and/or hetero atoms selected from the group consisting of O, S, Si and P and/or groups selected from the group consisting of —NH and —NR with R representing a linear, branched or cyclic, saturated or unsaturated hydrocarbon-based radical containing 1 to 20, preferably 1-15 and better still 1 to 6 carbon atoms, optionally comprising 1 to 5 hetero atoms selected from the group consisting of N, O, S, Si and P;
   (ii) the indolinoaryl groups of formula (III): 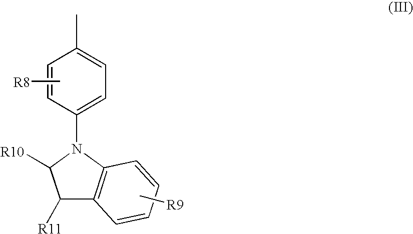
   in which R10 and R11 represent, independently of each other, a group selected from the group consisting of (i) linear, branched or cyclic, saturated or unsaturated hydrocarbon-based groups containing 1 to 30, preferably 1-18 and better still 1 to 12, or even 1-6, carbon atoms, optionally comprising 1 to 5 hetero atoms selected from the group consisting of N, O, S, Si and P, and/or optionally halogenated or perhalogenated, especially with F, Br and/or Cl; (ii) halogen atoms, and especially F, Br and/or Cl; (iii) —CN (nitrile), —COOH (carboxylate) or —NO₂ (nitro) groups; (iv) a hydrogen atom; (v) a group selected from the group consisting of —C(O)NR2R3, —NR2R3, —OR4 and —SR4 with R2, R3 and R4 having the meanings given above; (vi) the radicals R10 and R11 together possibly forming a saturated or unsaturated hydrocarbon-based ring containing in total 5 to 8 atoms (including the atoms of the indoline ring), the atoms being selected from the group consisting of C, O, S and/or NR with R representing H or a linear, branched or cyclic, saturated or unsaturated hydrocarbon-based radical containing 1 to 20 or even 1-12 carbon atoms, optionally comprising 1 to 5 hetero atoms selected from the group consisting of N, O, S, Si and P,
   (iii) the groups of formula (IV): 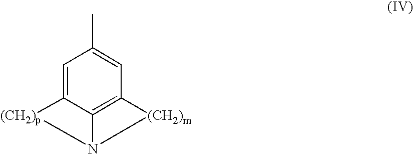
   in which m and p are, independently of each other, integers ranging from 2 to 5;
   (iv) the unsaturated cyclic aminoaryl groups of formula (Va), (Vb) or (Vc): 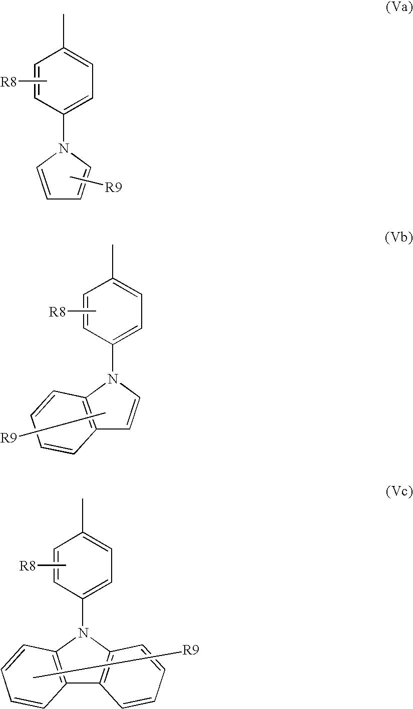
   in which R8 and R9 represent, independently of each other, a group selected from the group consisting of (i) linear, branched or cyclic, saturated or unsaturated hydrocarbon-based groups containing 1 to 30, preferably 1-18 and better still 1 to 12, or even 1-6, carbon atoms, optionally comprising 1 to 5 hetero atoms selected from the group consisting of N, O, S, Si and P, and/or optionally halogenated or perhalogenated, especially with F, Br and/or Cl; (ii) halogen atoms, especially F, Br and/or Cl; (iii) —CN (nitrile), —COOH (carboxylate) or —NO₂ (nitro) groups; (iv) a hydrogen atom; (v) a group selected from the group consisting of —C(O)NR2R3, —NR2R3, —OR4 and —SR4 with R2, R3 and R4 having the meanings given above;
   (v) a linear, branched or cyclic, saturated or unsaturated hydrocarbon-based group containing 1 to 30, preferably 2-18 and better still 3 to 12 carbon atoms, optionally comprising 1 to 5 hetero atoms selected from the group consisting of N, O, S, Si and P; and especially a group selected from the group consisting of —C₆H₄—CONR2R3, —C₆H₄—NR2R3 and —C₆H₄—OR4 with R2, R3 and R4 having the meanings given above;
R7 represents a group selected from the group consisting of:
(i) linear, branched or cyclic, saturated or unsaturated hydrocarbon-based groups containing 1 to 30, preferably 1-18 and better still 1 to 12 carbon atoms, optionally comprising 1 to 5 hetero atoms selected from the group consisting of N, O, S, Si and P, and/or optionally halogenated or perhalogenated, especially with F, Br and/or Cl;
(ii) halogen atoms, and especially F, Br and/or Cl;
(iii) —CN (nitrile), —COOH (carboxylate), —NO₂ (nitro), —N═N— (azo), ═NH (imino) or —CONH₂ (amide) groups;
(iv) a hydrogen atom;
(v) a group selected from the group consisting of —C(O)NR2R3, —NR2R3, —OR4 and —SR4 with R2, R3 and R4 having the meanings given above;
(vi) the radical R7 also possibly forming, with one of the bonds “i”, “j”, “k” or “g,h” taken with the radical R1, or “f” taken with the radical R1, a saturated hydrocarbon-based ring containing in total 3 to 8, preferably 4 to 7 and better still 5 or 6 carbon atoms, optionally comprising 1 to 5 hetero atoms selected from the group consisting of N, O, S, Si and P;
R′1 represents a group selected from the group consisting of:
(i) a hydrogen atom;
(ii) a linear, branched or cyclic, saturated or unsaturated hydrocarbon-based group containing 1 to 30, preferably 1-18 and better still 1 to 12 carbon atoms, optionally comprising 1 to 5 hetero atoms selected from the group consisting of N, O, S, Si and P, and/or optionally halogenated or perhalogenated, especially with F, Br and/or Cl;
(iii) a group selected from the group consisting of —C(O)NR2R3, —NR2R3, —OR4 and —SR4, with R2, R3 and R4 having the meanings given above;
R′2 represents a group selected from the group consisting of:
(i) linear, branched or cyclic, saturated or unsaturated hydrocarbon-based groups containing 1 to 30, preferably 1-18 and better still 1 to 12 carbon atoms, optionally comprising 1 to 5 hetero atoms selected from the group consisting of N, O, S, Si and P, and/or optionally halogenated or perhalogenated, especially with F, Br and/or Cl;
(ii) halogen atoms, especially F, Br and/or Cl;
(iii) —CN (nitrile), —COOH (carboxylate), —NO₂ (nitro), —N═N— (azo), ═NH (imino) or —CONH₂ (amide) groups;
(iv) a hydrogen atom;
(v) a group selected from the group consisting of —C(O)NR2R3, —NR2R3, —OR4 and —SR4, with R2, R3 and R4 having the meanings given above.

Preferably, the organic dyes according to the invention correspond to one of the formulae (Ia) and (IIa) below: 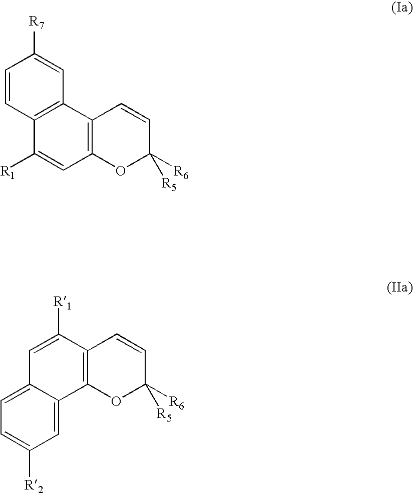
in which R1, R5, R6, R7, R′1 and R′2 are defined as above.

Preferably, R1 represents a hydrogen atom; a hydrocarbon-based ring with one of the bonds “f” or 20 “gh” and the radical R7; or a group selected from the group consisting of —COOR4, —NR2R3, —OR4 and —SR4, in which:
R2 and R3 either represent, independently of each other, a linear, branched or cyclic, saturated or unsaturated hydrocarbon-based group containing 1 to 20, preferably 1-12 and better still 1 to 6 carbon atoms, optionally comprising 1 to 5 hetero atoms selected from the group consisting of N, O, S, Si and P, or, taken together with the nitrogen atom to which they are attached, form a saturated or unsaturated hydrocarbon-based heterocycle containing 3-10 and preferably 4-6 carbon atoms and optionally 1-5 other hetero atoms selected from the group consisting of N, O, S, Si and P, the ring optionally being substituted with at least one linear, branched or cyclic, saturated or unsaturated hydrocarbon-based radical containing 1 to 20, preferably 1-15 and better still 1 to 6 carbon atoms, optionally comprising 1 to 5 hetero atoms selected from the group consisting of N, O, S, Si and P;
R4 represents a linear, branched or cyclic, saturated or unsaturated hydrocarbon-based group containing 1 to 20, preferably 1-15 and better still 1 to 6 carbon atoms, optionally halogenated or perhalogenated, especially with F, Br and/or Cl, and/or optionally comprising 1 to 5 hetero atoms selected from the group consisting of N, O, S, Si and P.

Preferably, R5 and R6 represent, independently of each other, a group selected from the group consisting of:
the saturated cyclic aminoaryl groups of formula (IIa) or (IIb): 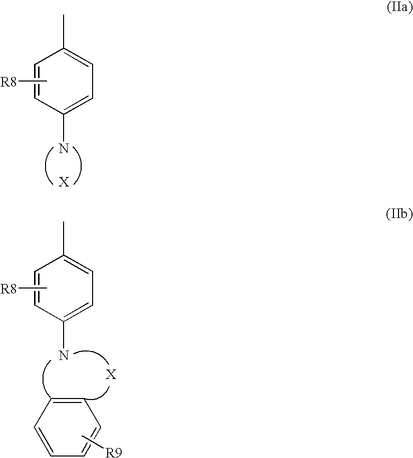
in which the ring comprising N and X is a saturated ring which contains in total 3 to 30, preferably 4-10 and better still 5, 6 or 7 atoms, including nitrogen, the rest being carbon atoms and/or hetero atoms selected from the group consisting of O, S, Si and P and/or groups selected from the group consisting of —NH and —NR with R representing a linear, branched or cyclic, saturated or unsaturated hydrocarbon-based radical containing 1 to 20, preferably 1-15 and better still 1 to 6 carbon atoms, optionally comprising 1 to 5 hetero atoms selected from the group consisting of N, O, S, Si and P;
a linear, branched or cyclic, saturated or unsaturated hydrocarbon-based group containing 1 to 30, preferably 2-18 and better still 3 to 12 carbon atoms, optionally comprising 1 to 5 hetero atoms selected from the group consisting of N, O, S, Si and P; and especially a group selected from the group consisting of —C₆H₄—CONR2R3, —C₆H₄—NR2R3 and —C₆H₄—OR4 with R2, R3 and R4 having the meanings given above.

Preferably, R7 represents a group selected from the group consisting of:
(i) linear, branched or cyclic, saturated or unsaturated hydrocarbon-based groups containing 1 to 30, preferably 1-18 and better still 1 to 12 carbon atoms, optionally comprising 1 to 5 hetero atoms selected from the group consisting of N, O, S, Si and P, and/or optionally halogenated or perhalogenated, especially with F, Br and/or Cl;
(ii) halogen atoms, especially F, Br and/or Cl;
(iii) —CN (nitrile), —COOH (carboxylate), —NO₂ (nitro), —N═N— (azo), ═NH (imino) or —CONH₂ (amide) groups;
(iv) a hydrogen atom;
(v) a group selected from the group consisting of —NR2R3, —OR4 and —SR4, with R2, R3 and R4 having the meanings given above;
(vi) the radical R7 also possibly forming, with one of the bonds “i”, “j”, “k” or “g,h” taken with the radical R1, or “f” taken with the radical R1, a saturated hydrocarbon-based ring containing in total 3 to 8, preferably 4 to 7 and better still 5 or 6 carbon atoms, optionally comprising 1 to 5 hetero atoms selected from the group consisting of N, O, S, Si and P.

Preferably, in formula (I) or (Ia), at least one of the groups R1 or R7 is other than hydrogen.

Preferably, in formula (I) or (Ia), at least one of the groups R5 or R6 is other than a phenyl radical (—C₆H₅).

Preferably also, in formula (I) or (Ia), at least one of the groups R1, R7, R5 or R6 comprises a nitrogen atom.

Preferably, in formula (I) or (Ia), at least one of the groups R5 or R6 represents a substituted phenyl radical.

Preferably, R′1 represents hydrogen or a linear, branched or cyclic, saturated or unsaturated hydrocarbon-based group containing 1 to 30, preferably 1-18 and better still 1 to 12 carbon atoms, optionally comprising 1 to 5 hetero atoms selected from the group consisting of N, O, S, Si and P, and/or optionally halogenated or perhalogenated, especially with F, Br and/or Cl.

Preferably, R′2 represents hydrogen or a group selected from the group consisting of —NO₂, —NR2R3 and —C(O)NR2R3, in which R2 and R3 either represent, independently of each other, a linear, branched or cyclic, saturated or unsaturated hydrocarbon-based group containing 1 to 20, preferably 1-12 and better still 1 to 6 carbon atoms, optionally comprising 1 to 5 hetero atoms selected from the group consisting of N, O, S, Si and P; or, taken together with the nitrogen atom to which they are attached, form a saturated or unsaturated hydrocarbon-based heterocycle containing 3-10 and preferably 4-6 carbon atoms and optionally 1-5 other hetero atoms selected from the group consisting of N, O, S, Si and P, the ring optionally being substituted with at least one linear, branched or cyclic, saturated or unsaturated hydrocarbon-based radical containing 1 to 20, preferably 1-15 and better still 1 to 6 carbon atoms, optionally comprising 1 to 5 hetero atoms selected from the group consisting of N, O, S, Si and P.

Mention may be made more particularly of the organic dyes of formula (I) or (Ia) for which:
R1 represents hydrogen; or a group —COOR with R being a saturated hydrocarbon-based radical containing 1 to 12 and preferably 1-6 carbon atoms, and especially a methyl or ethyl radical; or a group 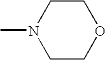
   and/or
R5 and R6 represent, independently of each other, either (i) a group of formula (IIa): 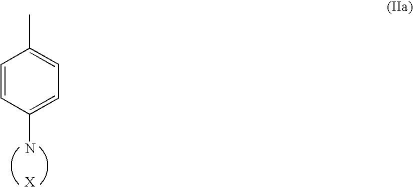
in which the ring comprising N and X is a saturated ring containing in total 4 to 7 atoms and especially 5 or 6 atoms, including nitrogen, and especially 3-5 carbon atoms and 0-1 oxygen atom; and in particular a group of formula: 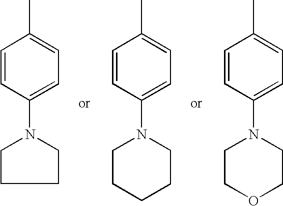
or (ii) a linear, branched or cyclic, saturated or unsaturated hydrocarbon-based group containing 5 to 14 and preferably 6 to 10 carbon atoms, optionally comprising 1 or 2 hetero atoms selected from the group consisting of N, O and S; in particular a group 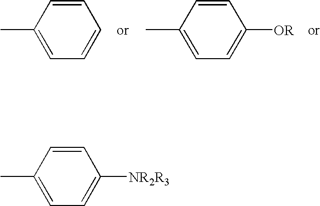
in which R is a saturated hydrocarbon-based radical containing 1 to 12 and preferably 1-6 carbon atoms, and especially a methyl or ethyl radical; and R2 and R3 are, independently of each other, a linear, branched or cyclic, saturated or unsaturated hydrocarbon-based group containing 1 to 20, preferably 1-15 and better still 1 to 6 carbon atoms, optionally comprising 1 to 5 hetero atoms selected from the group consisting of N, O, S, Si and P; and/or
R7 represents a hydrogen atom or a group —NR2R3, with R2 and R3 representing, independently of each other, a linear or branched, saturated hydrocarbon-based group containing 1 to 12 and preferably 1-6 carbon atoms, and especially a methyl and/or ethyl group.

Mention may also be made of the organic dyes of formula (II) or (IIa) for which:
R′1 represents hydrogen or a group —COOR with R being a saturated hydrocarbon-based radical containing 1 to 12 and preferably 1-6 carbon atoms, and especially a methyl or ethyl radical; and/or
R5 and R6 represent, independently of each other, either (i) a group of formula (IIa): 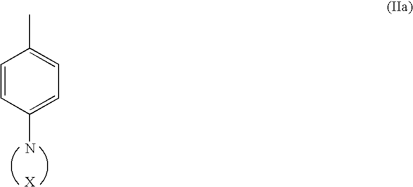
in which the ring comprising N and X is a saturated ring containing in total 4 to 7 atoms and especially 5 to 6 atoms, including nitrogen, and especially 4-5 carbon atoms and 0-1 oxygen atom; and in particular a group of formula: 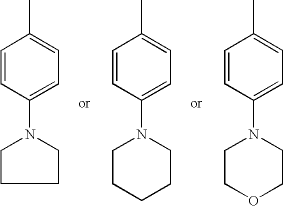
or (ii) a linear, branched or cyclic, saturated or unsaturated hydrocarbon-based group containing 5 to 14 and preferably 6 to 10 carbon atoms, optionally comprising 1 or 2 hetero atoms selected from the group consisting of N, O and S;
in particular a group: 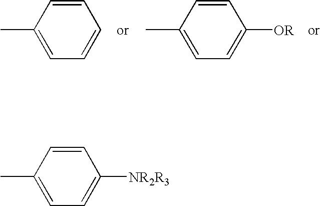
in which R is a saturated hydrocarbon-based radical containing 1 to 12 and preferably 1-6 carbon atoms, and especially a methyl or ethyl radical; and R2 and R3 are, independently of each other, a linear, branched or cyclic, saturated or unsaturated hydrocarbon-based group containing 1 to 20, preferably 1-15 and better still 1 to 6 carbon atoms, optionally comprising 1 to 5 hetero atoms selected from the group consisting of N, O, S, Si and P; and/or
R′2 represents hydrogen or a group —NR′R″, with R′ and R″, which may be identical or different, representing a linear or branched, saturated hydrocarbon-based group containing 1 to 12 and preferably 1-6 carbon atoms, and especially a methyl and/or ethyl group; or a group

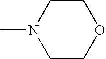

Among these organic dyes that may be mentioned are those described in patent applications WO 94/22850, WO 98/45281 and WO 00/18755.

Mention may be made especially of the following compounds, which are more particularly preferred:
3,3-di(4-methoxyphenyl)-6-morpholino-3H-naphtho-[2,1-b]pyran of formula: 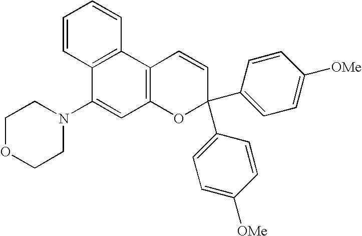
3-phenyl-3-(4-morpholinophenyl)-6-morpholino-3H-naphtho[2,1-b]pyran of formula: 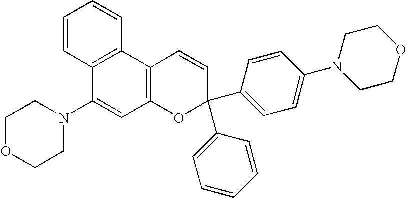
3-phenyl-3-(4-piperidinophenyl)-6-morpholino-3H-naphtho[2,1-b]pyran of formula: 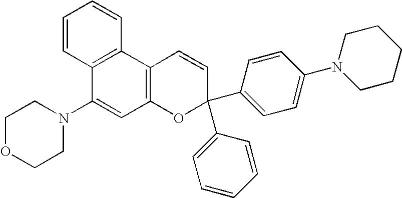
3-phenyl-3-(4-piperidinophenyl)-6-methoxycarbonyl 9-N-dimethyl-3H-naphtho[2,1-b]pyran of formula: 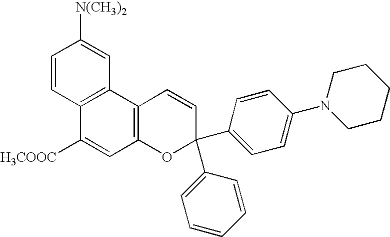
2-phenyl-2-(4-piperidinophenyl)-5-methoxycarbonyl-9-N-dimethyl-2H-naphtho[1,2-b]pyran of formula: 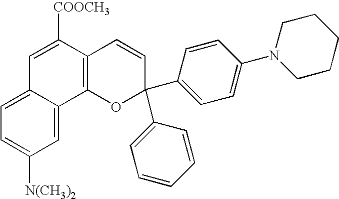
   mixtures thereof.

The organic dyes according to the invention are preferably included, alone or as a mixture, in an amount of from 0.001% to 20% by weight, especially from 0.005% to 10% by weight, preferably 0.01% to 4.8% by weight, better still from 0.05% to 3% by weight, preferentially from 0.1% to 2% by weight and especially 0.15-1% by weight, or even 0.2-0.8% by weight, relative to the total weight of the cosmetic composition.

Preferably, these organic dyes are photochromic, i.e. they have a ΔE value of greater than or equal to 5, preferably greater than or equal to 10, better still greater than or equal to 25 and even better still greater than or equal to 35, or even greater than or equal to 45; the ΔE value is measured in the manner defined before the examples.

Advantageously, the organic dyes according to the invention are soluble in the oily phase of the composition.

The term “soluble” means that the dyes form a uniform mixture, free of particles that are apparent to the naked eye, at 25° C. and 1 atmosphere, in a proportion of 0.05% by weight in the oily phase.

The composition according to the invention also comprises an oily phase that may comprise one or more cosmetically acceptable oils.

According to the invention, the expression “cosmetically acceptable oil” means any fatty substance that is liquid at 25° C. and 1 atmosphere, and preferably having a molecular weight of greater than or equal to 160, especially between 170 and 10⁶, or even between 200 and 5×10⁵, which is compatible with application to the skin, mucous membranes (lips) and/or the integuments (nails, eyelashes, eyebrows or hair).

Preferably, the oily phase is macroscopically homogeneous, i.e. homogeneous to the naked eye.

In a first particular embodiment of the invention, the oily phase is polar and may comprise mainly, or even exclusively, one or more polar oils (relatively or definitely polar) as a mixture, which may thus represent 5% to 100% by weight, especially 10-90%, or even 15-60% and better still 20-50% by weight, relative to the total weight of the oily phase. According to this preferred embodiment, the polar oily phase preferably has a mean solubility parameter δₐ according to the Hansen solubility space, at 25° C., of greater than or equal to 5.0 (J/cm³)^{1/2} especially greater than or equal to 5.3, or even greater than or equal to 5.5 and better still greater than or equal to 6.0 (J/cm³)^{1/2}, or even greater than or equal to 7.0 (J/cm³)^{1/2}.

The term “polar oil” means an oil composed of chemical compounds comprising at least one polar group.

Polar groups are well known to those skilled in the art: they may especially be groups of alcohol, ester or carboxylic acid type

In particular, the polar oils according to the invention may be defined as having a mean solubility parameter δₐ according to the Hansen solubility space, at 25° C., of: δₐ ≧5.0 (J/cm³)^{1/2}.

The polar oils comprise relatively polar oils that have a mean solubility parameter at 25° C. of: 5.0≦δₐ≦7.0 (J/cm³)^{1/2}, and the definitely polar oils, which have a mean solubility parameter at 25° C. of: δₐ>7.0 (J/cm³)_{1/2}.

Similarly, for the purposes of the invention, the apolar oils have a mean solubility parameter δₐ according to the Hansen solubility space, at 25° C., of: 0≦δₐ≦5.0 (J/Cm³)^{1/2}.

For the purposes of the invention, the apolar oils comprise the definitely apolar oils (δₐ=0) and the sparingly polar oils, which have a mean solubility parameter at 25° C. of: 0<δₐ<5.0 (J/cm³)^{1/2}.

Thus, the higher the value of δₐ, the higher the polarity of the oil.

The definition and calculation of the solubility parameters in the three-dimensional Hansen solubility space are described in the article by C. M. Hansen: “The three dimensional solubility parameters”, J. Paint Technol. 39, 105 (1967).

According to this Hansen space:
δ_{D} characterizes the London dispersion forces derived from the formation of dipoles induced during molecular impacts;
δₚ characterizes the Debye interaction forces between permanent dipoles and also the Keesom interaction forces between induced dipoles and permanent dipoles;
δₕ characterizes the specific interaction forces (such as hydrogen bonding, acid/base, donor/acceptor, etc.);
δₐ is determined by the equation: δₐ=(δₚ²+δₕ²)^{1/2}.

The parameters δₚ, δₕ, δ_{D} and δₐ are expressed in (J/cm³)^{1/2}.

When the oily phase is a mixture of different oils, the solubility parameters of the mixture are determined from those of the compounds taken separately, according to the following relationships:

$${δ}_{Dmixt} = {Σ}_{i} x i {δ}_{Di} ; {δ}_{pmixt} = {Σ}_{i} xi {δ}_{pi} and {δ}_{hmixt} = {Σ}_{i} xi {δ}_{hi}$$

δₐₘᵢₓₜ=(δ²ₚₘᵢₓₜ+δ²ₕₘᵢₓₜ)^{1/2}
in which xi represents the volume fraction of the compound i in the mixture.

It is within the capability of a person skilled in the art to determine the amounts of each oil in order to obtain an oily phase that satisfies the desired criteria.

According to a second particular embodiment of the invention, the oily phase is apolar and may comprise 5% to 100% by weight, especially 10-90%, or even 15-60% and better still 20-50% by weight of one or more apolar (apolar or sparingly polar) oils; it has a mean solubility parameter δₐ according to the Hansen solubility space, at 25° C., of less than 5.0, especially less than or equal to 4.9, better still less than or equal to 4.5 and even better still less than or equal to 4.0 (J/cm³)^{1/2}.

The polar or apolar oily phase may comprise one or more oils, which may themselves be polar or apolar, and volatile or non-volatile, and preferably hydrocarbon-based.

These oils may be chosen, alone or as a mixture, from volatile or non-volatile oils of animal, plant, mineral or synthetic origin.

The term “volatile oils” means oils that have, at 25° C., a vapour pressure of between 0.02 and 300 mmHg (i.e. 2.66 to 40 000 Pa). Preferably, volatile oils with a flash point of about 30-100° C. are used.

Mention may be made especially of:
animal or plant oils formed from fatty acid esters of polyols, in particular liquid triglycerides, for example sunflower oil, maize oil, soybean oil, marrow oil, grapeseed oil, sesame seed oil, hazelnut oil, apricot oil, almond oil or avocado oil; fish oils or glyceryl tricaprocaprylate, or oils of plant or animal origin of formula R₁COOR₂ in which R₁ represents a higher fatty acid residue containing from 7 to 19 carbon atoms and R₂ represents a branched hydrocarbon-based chain containing from 3 to 20 carbon atoms, for example Purcellin oil; liquid paraffin, liquid petroleum jelly, beauty-leaf oil, macadamia oil, rapeseed oil, coconut oil, groundnut oil, palm oil, castor oil, jojoba oil, olive oil or cereal germ oil; shea butter oil; perhydrosqualene;
synthetic esters and ethers, especially of fatty acids, for instance the oils of formula R₁COOR₂ in which R₁ represents a higher fatty acid residue containing from 7 to 29 carbon atoms and R₂ represents a hydrocarbon-based chain containing from 3 to 30 carbon atoms, for instance isopropyl myristate, 2-ethylhexyl palmitate, 2-octyldodecyl stearate, 2-octyldodecyl erucate, isononyl isononanoate or isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate or diethylene glycol diisononanoate; pentaerythritol esters, for instance pentaerythrityl tetraisostearate; esters of the type such as tridecyl trimellitate;
fatty alcohols containing from 12 to 26 carbon atoms, for instance octyldodecanol, 2-butyloctanol, 2-hexyl-decanol, 2-undecylpentadecanol or oleyl alcohol;
linear or branched hydrocarbons of mineral or synthetic origin, such as liquid paraffins and derivatives thereof, petroleum jelly, polydecenes, and hydrogenated polyisobutene such as parleam; isoparaffins, for instance isohexadecane and isodecane;
glycerides and especially acetylglycerides or triglycerides of fatty acids containing 4 to 10 carbon atoms, for instance heptanoic, octanoic and capric/caprylic acid triglycerides.

Among the polar oils that are particularly preferred, mention may be made of octyldodecanol, hexyldecanol, octyldecanol, oleyl alcohol, castor oil, diisostearyl malate, glyceryl triheptanoate, glyceryl trioctanoate, capric/caprylic acid triglyceride, triisononanoin and tridecyl trimellitate, and mixtures thereof.

Among the apolar oils that are particularly preferred, mention may be made of aliphatic hydrocarbons, especially of C6-C40, for instance volatile liquid paraffins, such as isohexadecane or isododecane, or non-volatile liquid paraffins, and derivatives thereof; petroleum jelly, hydrogenated or non-hydrogenated polydecenes, hydrogenated polyisobutene such as parleam oil, squalane, polybutylenes and isononyl isononanoate; fluoro oils and especially perfluoro oils, and mixtures thereof.

Mention may be made especially of the following oils:

| | δₐ (J/cm³)^{1/2} |
|---|---|
| Castor oil | 9.09 |
| 2-Hexyldecanol | 8.55 |
| Oleyl alcohol | 8.17 |
| Octyldodecanol | 7.69 |
| Heptanoic acid triglyceride | 7.29 |
| Diisostearyl malate | 7.19 |
| Octanoic acid triglyceride | 6.87 |
| Capric/caprylic acid triglyceride | 6.69 |
| Triisononanoin | 6.54 |
| Tridecyl trimellitate | 5.35 |
| Isononyl isononanoate | 4.87 |
| Hydrogenated polyisobutene | 0 |
| Isododecane | 0 |

According to a third particular embodiment of the invention, the oily phase may comprise at least one phenylsilicone oil especially of formula (VI), or a mixture of such oils: 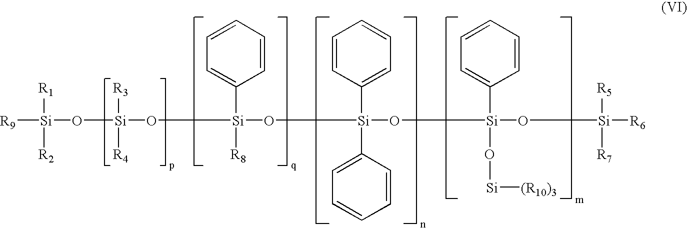
in which
—R1 to R10, independently of each other, are saturated or unsaturated, linear, cyclic or branched C1-C30 hydrocarbon-based radicals,
m, n, p and q are, independently of each other, integers between 0 and 900, with the proviso that the sum “m+n+q” is other than 0.

Preferably, the sum “m+n+q” is between 1 and 100.

Preferably, the sum “m+n+p+q” is between 1 and 900 and better still between 1 and 800.

Preferably, q is equal to 0.

Preferably, the phenylsilicone oils are of formula (VII): 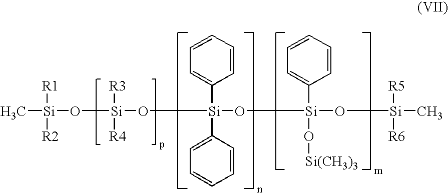
in which:
R1 to R6, independently of each other, are saturated or unsaturated, linear, cyclic or branched C1-C30 hydrocarbon-based radicals,
m, n and p are, independently of each other, integers between 0 and 100, with the proviso that the sum “n+m” is between 1 and 100.

Preferably, R1 to R6, independently of each other, represent a saturated linear or branched C1-C30 and especially C1-C12 hydrocarbon-based radical, and in particular a methyl, ethyl, propyl or butyl radical.

R1 to R6 may especially be identical, and may also be a methyl radical.

Preferably, it is possible to have m=1 or 2 or 3, and/or n=0 and/or p=0 or 1.

The phenylsilicone oils may be selected from the group consisting of phenyl trimethicones, phenyl dimethicones, phenyl trimethylsiloxy diphenylsiloxanes, diphenyl dimethicones and diphenyl methyldiphenyl trisiloxanes, and mixtures thereof.

Preferably, the mass-average molecular weight of the phenylsilicone oil is between 500 and 10 000.

Mention may be made especially of the phenyl-silicone oils of formula (VI) with a viscosity at 25° C. of between 3 and 1 500 mm²/s (i.e. 3 to 1 500 cSt), preferably with a viscosity of between 5 and 1 000 mm²/s (i.e. 5 to 1 000 cSt). Mention may be made especially of phenyl trimethicones such as DC556 from Dow Corning (22.5 cSt), the oil Silbione 70663V30 from Rhône Poulenc (28 cSt), the oil Abil AV8853 from Goldschmidt (4-6 cSt), or diphenyl dimethicones such as the Belsil oils, especially Belsil PDM1000 (1 000 cSt), Belsil PDM 200 (200 cSt) and Belsil PDM 20 (20 cSt) from Wacker. The values in parentheses represent the viscosities at 25° C.

In this third embodiment, the oily phase may comprise, in addition to the phenylsilicone oil, one or more other additional cosmetically acceptable oils, which may be polar or apolar, volatile or non-volatile, and silicone-based or hydrocarbon-based.

Among the additional oils that may be used, mention may be made, alone or as a mixture, of polar or apolar oils of animal, plant, mineral or synthetic origin, such as those mentioned above.

Among the additional oils that may also be mentioned are volatile silicone oils such as:
cyclic volatile silicones containing from 3 to 8 and preferably from 4 to 6 silicon atoms. These are, for example, cyclotetradimethylsiloxane, cyclopentadi-methylsiloxane or cyclohexadimethylsiloxane,
cyclocopolymers of the dimethylsiloxane/methylalkylsiloxane type, such as Silicone FZ 3109 sold by the company Union Carbide, which is a dimethylsiloxane/methyloctylsiloxane cyclocopolymer,
linear volatile silicones containing from 2 to 9 silicon atoms. These are, for example, hexamethyl-disiloxane, hexylheptamethyltrisiloxane or octylhepta-methyltrisiloxane.

It is also possible to use non-volatile silicone oils such as:
poly(C₁-C₂₀)alkylsiloxanes and especially those containing trimethylsilyl end groups, preferably those with a viscosity of less than 0.06 m²/s; mention may be made especially of linear or cyclic polydimethyl-siloxanes, optionally comprising an alkyl or alkoxy group, which is pendent or at the end of a chain; alkylmethylpolysiloxanes such as cetyl dimethicone (CTFA name),
silicones modified with optionally fluorinated aliphatic and/or aromatic groups, or with functional groups such as hydroxyl, thiol and/or amine groups.

In this 3rd particular embodiment, the composition preferably comprises the phenylsilicone oil(s) in an amount of between 1-99% by weight, especially 5-90% by weight, preferably 7-50% by weight, or even 13-40% by weight, relative to the total weight of the cosmetic composition.

The additional oil(s) is(are) then present in an amount preferably of between 0.1-50% by weight, especially 4-30% by weight and preferably 10-25% by weight, relative to the total weight of the cosmetic composition.

Whatever the embodiment, the oily phase is preferably included in an amount of from 1% to 99% by weight, especially 10% to 90% by weight and preferably 15% to 80% by weight, relative to the total weight of the cosmetic composition.

The composition according to the invention may also comprise fatty substances other than the above oils, which may be chosen by a person skilled in the art on the basis of his general knowledge, so as to give the final composition the desired properties, for example in terms of consistency and/or texture.

These additional fatty substances may be waxes, gums and/or pasty fatty substances of animal, plant, mineral or synthetic origin, and also mixtures thereof.

For the purposes of the present invention, a wax is a lipophilic fatty compound, which is solid at room temperature (25° C.), with a reversible solid/liquid change of state, which has a melting point of greater than 40° C. and which may be up to 200° C., which generally has a hardness of greater than 0.5 MPa and which has an anisotropic crystal organization in the solid state.

Mention may be made especially of waxes of animal, plant, mineral or synthetic origin, such as micro-crystalline waxes, paraffin wax, petrolatum, petroleum jelly, ozokerite or montan wax; beeswax, lanolin wax and derivatives thereof; candelilla wax, ouricury wax, carnauba wax, Japan wax, cocoa butter, cork fibre wax or sugar cane wax, lignite wax, rice bran wax, fir tree wax and cotton wax; hydrogenated oils with a melting point of greater than (about) 40° C., for instance hydrogenated jojoba oil; fatty esters and glycerides that are solid at 25° C.; polyethylene waxes and the waxes obtained by Fischer-Tropsch synthesis; hydrogenated oils that are solid at 25° C.; lanolins. When the oily phase comprises silicone oils, use may also be made of silicone waxes such as alkyl- or alkoxydimethicones with an alkyl or alkoxy chain containing 10-45 carbon atoms; polysiloxane esters, especially polydimethylsiloxane esters, which are solid at 30° C., the ester chain of which contains 10-45 carbon atoms, and/or silicone resins.

The pasty fatty substances generally have a melting point of between 25 and 60° C. and preferably between 30 and 45C, and/or a hardness ranging from 0.001 to 0.5 MPa and preferably between 0.005 and 0.4 MPa.

Mention may be made especially of lanolins and derivatives thereof, or cholesterol esters.

These additional fatty substances may be present in an amount of 0.1-50% by weight, especially 3-40% by weight and better still 5-30% by weight, relative to the total weight of the composition.

The composition according to the invention may be anhydrous, or may also comprise an aqueous phase, which may comprise water, a floral water such as cornflower water and/or a mineral water such as eau de Vittel, eau de Lucas or eau de La Roche Posay.

The aqueous phase may comprise from 0.1% to 14% by weight, relative to the total weight of the aqueous phase, of a C₂-C₆ monoalcohol, for instance ethanol, propanol, butanol, isopropanol or isobutanol.

The composition according to the invention may optionally comprise a surfactant, especially when it is in the form of an emulsion, preferably in an amount of from 0.01% to 30% by weight relative to the total weight of the composition.

Mention may be made, alone or as a mixture, of alkali metal salts, ammonium salts, amine salts or amino alcohol salts of the following compounds: alkyl sulphates, alkyl ether sulphates, alkylamide sulphates and ether sulphates, alkylarylpolyether sulphates, monoglyceride sulphates, alkyl sulphonates, alkylamide sulphonates, alkylarylsulphonates, α-olefin sulphonates, paraffin sulphonates, alkyl sulpho-succinates, alkyl ether sulphosuccinates, alkylamide sulphosuccinates, alkyl sulphosuccinamates, alkyl sulphoacetates, alkylpolyglycerol carboxylates, alkyl phosphates/alkyl ether phosphates, acyl sarcosinates, alkyl polypeptidates, alkylamidopolypeptidates, acyl isethionates, alkyl laurates. The alkyl or acyl radical of all these compounds generally denotes a chain of 12 to 18 carbon atoms.

Mention may also be made of soaps and salts of fatty acids such as oleic acid, ricinoleic acid, palmitic acid, stearic acid and the acids of coconut oil or of hydrogenated coconut oil, and especially amine salts such as amine stearates; acyl lactylates in which the acyl radical contains 8-20 carbon atoms; polyglycol ether carboxylic acids.

Mention may also be made of polyethoxylated, polypropoxylated or polyglycerolated fatty alcohols, fatty alkylphenols and fatty acids, with a fatty chain containing from 8 to 18 carbon atoms; copolymers of ethylene oxide and of propylene oxide, condensates of ethylene oxide and of propylene oxide on fatty alcohols, polyethoxylated fatty amides, polyethoxylated fatty amines, ethanolamides, fatty acid esters of glycol, oxyethylenated or non-oxyethylenated fatty acid esters (stearate or oleate) of sorbitan, fatty acid esters of sucrose, fatty acid esters of polyethylene glycol (polyethylene glycol monostearate or monolaurate); phosphoric triesters, fatty acid esters of glucose derivatives; alkylpolyglycosides and alkyl-amides of amino sugars; the products of condensation of an α-diol, of a monoalcohol, of an alkylphenol, of an amide or of a diglycolamide with glycidol or a glycidol precursor.

Mention may also be made of trioleyl phosphate; fatty acid esters of polyols, for instance glyceryl or sorbitol mono-, di-, tri- or sesquioleates or stearates, and glyceryl or polyethylene glycol laurates; alkyl or alkoxy dimethicone copolyols with an alkyl or alkoxy chain that is pendent or at the end of the silicone skeleton, for example containing from 6 to 22 carbon atoms; polyoxyethylenated alkyl (lauryl, cetyl, stearyl or octyl) ethers and dimethicone copolyols.

The composition according to the invention may also comprise one or more thickeners, for example in preferred concentrations of from 0.01% to 6% by weight relative to the total weight of the composition. The thickener may be chosen, alone or as a mixture, from:
polysaccharide biopolymers, for instance xanthan gum, carob gum, guar gum, alginates, modified celluloses such as hydroxyethylcellulose, methylcellulose, hydroxypropylcellulose and carboxymethylcellulose, starch derivatives, cellulose ether derivatives containing quaternary ammonium groups, and cationic polysaccharides;
synthetic polymers, for instance polyacrylic acids such as polyglyceryl (meth)acrylate polymers such as Hispagel or Lubragel from the companies Hispano Qimica or Guardian, polyvinylpyrrolidone, polyvinyl alcohol, crosslinked polymers of acrylamide and of ammonium acrylate, such as PAS 5161 or Bozepol C from Hoechst; acrylate/octylacrylamide copolymers, such as Dermacryl from National Starch; polyacrylamide-based polymers, such as Sepigel 305 from SEPPIC, crosslinked polymers of acrylamide and of methacryloyloxyethyltrimethyl-ammonium chloride, such as Salcare SC 92 from Allied Colloids,
magnesium aluminium silicate;
optionally modified clays, for instance hectorites modified with a C₁₀ to C₂₂ fatty acid ammonium chloride, for instance hectorite modified with distearyldimethyl-ammonium chloride;
optionally modified silica;
galactomannans comprising one to six and better still from two to four hydroxyl groups per saccharide, substituted with a saturated or unsaturated alkyl chain, for instance guar gum alkylated with C₁ to C₆ and better still C₁ to C₃ alkyl chains and more particularly ethylated guar with a degree of substitution of 2 to 3, such as the product sold by the company Aqualon under the name N-Hance-AG;
cellulose derivatives such as ethylcellulose;
block copolymers, especially of “diblock” or “triblock” type, such as polystyrene/polyisoprene, polystyrene/polybutadiene, polystyrene/copoly(ethylene-butylene) or polystyrene/copoly(ethylene-propylene) such as those sold under the name “Kraton” by Shell Chemical;
polymers of polyamide type, for example comprising a polymer skeleton containing amide repeating units, and optionally at least one pendent fatty chain and/or at least one terminal chain, which are optionally functionalized, containing from 8 to 120 carbon atoms and being linked to these amide units, among which mention may be made of the products sold by the company Arizona Chemical under the names Uniclear 80 and Uniclear 100, which are a mixture of copolymers of a C₃₆ diacid condensed with ethylenediamine, with a weight-average molecular mass of about 6 000, and terminal ester groups resulting from the esterification of the remaining acid end groups with cetyl alcohol or stearyl alcohol, or mixtures thereof (also known as cetyl-stearyl alcohol).

Depending on the envisaged application, the composition may also comprise a film-forming polymer.

This is generally the case when it is desired to prepare a composition such as a nail varnish, mascara, eyeliner or lipstick.

The polymers may be dissolved or dispersed in the cosmetically acceptable medium. The polymers may be present in a content ranging from 0.01% to 40% by weight relative to the total weight of the composition.

The composition may also comprise a plasticizer selected from the group consisting of the usual plasticizers, which may be present in a content ranging from 0.1% to 40% by weight relative to the total weight of the composition.

The composition may also comprise a particulate phase, which may comprise pigments and/or nacres and/or fillers usually used in cosmetic compositions.

The term “pigments” should be understood as meaning white or colored, mineral or organic particles, intended to color and/or opacify the composition. The term “fillers” should be understood as meaning colorless or white, mineral or synthetic, lamellar or non-lamellar particles intended to give the composition body or rigidity, and/or to give the makeup softness, a matt effect and uniformity. The term “nacres” should be understood as meaning iridescent particles that reflect light, which are produced especially by certain molluscs in their shell or alternatively which are synthesized.

The pigments may be present in the composition in a proportion of from 0.01% to 25% by weight of the final composition and preferably in a proportion of from 3% to 10% by weight. They may be white or colored, mineral or organic, and of standard or nanometric size. They may be in the form of powder or of pigmentary paste.

Mention may be made of titanium oxide, zirconium oxide or cerium oxide, and also zinc oxide, iron oxide, or chromium oxide, ferric blue, chromium hydrate, carbon black, ultramarines (aluminosilicate polysulphides), manganese pyrophosphate and certain metal powders such as silver or aluminium powder. Mention may also be made of the D&C pigments and lakes commonly used to give the lips and the skin a makeup effect, which are calcium, barium, aluminium, strontium or zirconium salts. The nacres may be present in the composition in a proportion of from 0.01% to 20% by weight and preferably in a content of about from 3% to 10% by weight. Among the nacres that may be envisaged, mention may be made of natural nacre, mica coated with titanium oxide, with iron oxide, with natural pigment or with bismuth oxychloride, and also colored titanium mica. The fillers, which may be present in a proportion of from 0.01% to 60% by weight and preferably 3% to 10%, in the composition, may be mineral or synthetic, and lamellar or non-lamellar. Mention may be made of talc, mica, silica, kaolin, Nylon powder, polyethylene powder, Teflon, starch, boron nitride, polymer microspheres such as Expancel (Nobel Industrie), Polytrap (Dow Corning) and silicone resin microbeads (for example Tospearls from Toshiba), precipitated calcium carbonate, magnesium carbonate, magnesium hydrocarbonate and metal soaps derived from organic carboxylic acids containing from 8 to 22 carbon atoms.

The composition may also comprise a dye, especially a natural organic dye such as cochineal carmine, and/or a synthetic dye, such as haloacid dyes, azo dyes or anthraquinone dyes. Mention may also be made of mineral dyes such as copper sulphate or iron sulphate. Mention may also be made of Sudan brown, Sudan red and annatto, and also beetroot juice, carotene and methylene blue. The dye may be present in the composition, alone or as a mixture, in a proportion of from 0.001% to 15% by weight, preferably 0.01% to 5% by weight and especially from 0.1% to 2% by weight relative to the total weight of the composition.

The composition may also comprise a photochromic compound other than the photochromic organic dyes of formula (I) or (II). Mention may be made especially of mineral photochromic compounds, and more particularly doped aluminosilicates such as halogen-doped sodalite, or metal oxides or hydrates, such as titanium oxides rendered photochromic with the aid of a metal selected from the group consisting of iron, chromium, copper, nickel, manganese, cobalt or molybdenum, in elemental form or in the form of a salt such as a sulphate, a chlorate, a nitrate or an acetate. The photochromic compound may be incorporated into the composition in an amount of 0.001-20% by weight and preferably in an amount of 0.1-10% by weight relative to the total weight of the composition.

The composition may also comprise a UV-screening agent, which may be incorporated into the composition in an amount of 0.01-20% by weight and preferably in an amount of 0.1-10% by weight relative to the total weight of the composition. Among the sunscreens that may be used, mention may be made especially of the compounds belonging to the following families: para-aminobenzoics; salicylates; dibenzoyl-methanes; cinnamates; β,β′-diphenylacrylate derivatives; benzophenones; benzylidenecamphors; phenylbenzimidazoles; triazines; phenylbenzotriazoles; anthranilics; imidazolines and/or benzalmalonates.

The composition may also comprise any additive usually used in cosmetics, such as antioxidants, fragrances, essential oils, preserving agents, lipophilic or hydrophilic cosmetic active agents, moisturizers, vitamins, sphingolipids, self-tanning agents such as DHA, optical brighteners, antifoams and sequestering agents.

Needless to say, a person skilled in the art will take care to select the optional additional compounds, and/or the amount thereof, such that the advantageous properties of the composition according to the invention are not, or are not substantially, adversely affected by the envisaged addition.

The composition according to the invention is advantageously photochromic, i.e. it has a ΔE value of greater than or equal to 5, preferably greater than or equal to 10, better still greater than or equal to 25 and even better still greater than or equal to 35, or even greater than or equal to 45; the ΔE value is measured in the manner defined before the examples.

The cosmetic composition according to the invention especially finds a particularly advantageous application in the field of making up the skin, semi-mucous membranes, mucous membranes and/or the integuments (nails, eyelashes, eyebrows, body hair and head hair).

The composition may comprise or may be in the form of a suspension or a dispersion especially of oil in water by means of vesicles; an optionally thickened or even gelled oily solution; an oil-in-water, water-in-oil or multiple emulsion; a gel or a mousse; an oily or emulsified gel; a dispersion of vesicles, which are especially lipid vesicles; a two-phase or multiphase lotion; a spray; a free, compact or cast powder; an anhydrous paste. This composition may have the appearance of a lotion, a cream, an ointment, a soft paste, a salve, a cast or a moulded solid especially in the form of a stick or a dish, or alternatively a compacted solid.

The cosmetic composition according to the invention may be in the form of a care and/or makeup product for body or facial skin, for the lips and for the hair, an antisun product or self-tanning product, or even a hair product. It finds a particular application in the field of lipsticks, foundations, makeup rouges, eyeshadows, free or compact powders, tinted creams, body makeup products, skin-coloring products, eyeliners and mascaras.

A subject of the invention is also a cosmetic process for treating a support selected from the group consisting of mucous membranes, semi-mucous membranes, the skin and/or the integuments, in which a cosmetic composition as defined above is applied to the support.

Another subject of the invention is a makeup and/or temporary coloring process for a support selected from the group consisting of mucous membranes, semi-mucous membranes, the skin and/or the integuments, in which a cosmetic composition as defined above is applied to the support.

The invention is illustrated in further detail in the examples that follow. These examples illustrate the invention and do not limit it.

Measurement of the ΔE

The L, a and b measurements were performed on a Leneta brand contrast card of reference Form 1A Penopac, for a composition spread to a thickness of 50 microns.

The reflection measurements were performed using a Minolta 3700D spectrocolorimeter.

The initial trichromatic coordinates of the composition before irradiation (L₀, a₀ and b₀) were thus determined. The composition was then subjected to a UV irradiation (flux of 2 mW/cm² of a UVA radiation) for two minutes and then, immediately after stopping the irradiation, the trichromatic coordinates after irradiation (L, a and b) were determined. Less than 5 seconds elapsed between the stopping of the irradiation and the determination of the new coordinates.

The ΔE was then calculated in the following manner:
ΔE=[(L−L₀)²+(a−a₀)²+(b−b₀) ²]^{1/2}

When the ΔE of a dye is measured rather than of a composition comprising it, the measurement is performed on a solution comprising 0.05% by weight of dye in diisostearyl malate. The measurement is then performed as indicated above.

### EXAMPLE 1

The solubility of a dye according to the invention in various silicone oils was studied. Several mixtures comprising 0.05% by weight of 3-phenyl-3-(4-piperidinophenyl)-6-morpholino-3H-naphtho[2,1-b]pyran (Reversacol Ruby from James Robinson) in various oils were thus prepared, and the mixture was then heated to 60° C. with stirring. The mixture of the dye with the oil was observed with the naked eye, at elevated temperature (60° C.) and then after cooling the heated mixture to 25° C.

The following results are obtained:

| | At elevated | |
|---|---|---|
| | temperature | After cooling to |
| Oil | (60° C.) | 25° C. |
| Phenyltrimethylsiloxytrisiloxane | Total dissolution | Virtually total |
| (viscosity: | | dissolution: a |
| 20 cSt) DC556 | | few undissolved |
| from Dow Corning | | grains remain |
| Polyphenyltrimethylsiloxydi- | Total dissolution | Virtually total |
| methylsiloxane | | dissolution: a |
| (viscosity: | | few undissolved |
| 1000 cSt) Belsil | | grains remain |
| PDM 1000 from | | |
| Wacker | | |
| Cyclopentadimethylsiloxane | Sedimentation of | Sedimentation of |
| (viscosity: | powder to the | the undissolved |
| 4 cSt) | bottom; virtually | dye (powder) to |
| | no dissolution; | the bottom; |
| | cloudy mixture | cloudy colorless |
| | (suspension of | solution with |
| | undissolved dye | particles in |
| | particles) | suspension |
| PDMS (viscosity: | Sedimentation of | Sedimentation of |
| 5 cSt) DC200 | powder to the | the undissolved |
| Fluid from Dow | bottom; virtually | dye (powder) to |
| Corning | no dissolution; | the bottom; |
| | cloudy mixture | cloudy colorless |
| | (suspension of | solution with |
| | undissolved dye | particles in |
| | particles) | suspension |
| Decamethyltetrasiloxane | Sedimentation of | Sedimentation of |
| (viscosity: | powder to the | the undissolved |
| 1.5 cSt) | bottom; virtually | dye (powder) to |
| | no dissolution; | the bottom; |
| | very slightly | transparent |
| | cloudy mixture | colorless |
| | (suspension of | solution with |
| | undissolved dye | particles in |
| | particles) | suspension |

It is thus found that the photochromic dye dissolves poorly at elevated temperature in non-phenylated oils, even though they are of low viscosity, which is more suited to good dissolution than a higher viscosity. On the other hand, the dissolution is complete at elevated temperature in the phenylated oils, irrespective of their viscosity.

After cooling to room temperature, the observations at elevated temperature are confirmed. The phenylsilicones allow a virtually complete dissolution of the photochromic dye after cooling to room temperature. The dissolution is, in this case also, much better than in the other silicone-based media, in which the dye is virtually undissolved (sedimentation of powder to the bottom of the flask).

Moreover, a pink coloration of the solution of the dye was observed in the phenylsilicones, and not for the other oils; this shows that there is also a solvatochromic effect for these oils.

### EXAMPLE 2

A solution comprising 0.05% by weight of 3-phenyl-3-(4-piperidinophenyl)-6-morpholino-3H-naphtho[2,1-b]pyran (Reversacol Ruby from James Robinson) in a phenylsilicone oil (DC556 from Dow Corning) was prepared. The mixture was then irradiated with UV (sunlight) for 2 minutes.

A very sharp change from an initial pink coloration of the mixture before irradiation to a strong orange-red coloration after irradiation was observed visually. This clearly confirms the existence of the photochromic effect.

### EXAMPLE 3

A composition was prepared, comprising:
0.05% by weight of 3-phenyl-3-(4-piperidinophenyl)-6-morpholino-3H-naphtho[2,1-b]pyran (Reversacol Ruby from James Robinson),
15% by weight of polyethylene wax (apolar wax),
qs 100% of phenylsilicone oil (DC556 from Dow Corning).

The L, a and b measurements were performed on a Leneta brand contrast card of reference Form 1A Penopac, for a composition spread to a thickness of 50 microns.

The reflection measurements were performed using a Minolta 3700D spectrocolorimeter.

The initial trichromatic coordinates of the composition before irradiation (L₀, a₀ and b₀) were thus determined.

The composition was then subjected to a UV irradiation (flux of 2 mW/cm² of a UVA radiation) for two minutes and then, immediately after stopping the irradiation, the trichromatic coordinates after irradiation (L, a and b) were determined. Less than 5 seconds elapsed between the stopping of the irradiation and the determination of the new coordinates.

The ΔE was then calculated in the following manner:
ΔE=[(L−L₀)²+(a−a₀)²+(b−b₀)²]^{1/2}

The following result is obtained: ΔE=37.38.

It is thus found that the photochromic effect still exists in a structured system, i.e. in the presence of a wax.

### EXAMPLE 4

A lipstick was prepared, comprising (% by weight):

| | |
|---|---|
| Reversacol Ruby | 0.05% |
| polyethylene wax | 15% |
| phenylsilicone oil (DC556) | 30% |
| parleam oil | qs 100% |

### EXAMPLE 5

The ΔE (before and after UV irradiation for 2 minutes) of various photochromic dyes according to the invention was determined, at 1% by weight in a white lipstick base comprising:

| | |
|---|---|
| 2-octyldodecanol | 0.5% |
| hectorite modified with distearyldimethyl- | 0.6% |
| ammonium chloride | |
| liquid lanolin | 27.2% |
| microcrystalline wax | 10.5% |
| polyglycerolated (3 mol) beeswax | 4.2% |
| acetylated lanolin | 6.7% |
| arara oil (oleic acid esters) | 13.5% |
| oxypropylenated (5 OP) lanolin wax | 6.7% |
| oleyl erucate | 13.5% |
| oleic-linoleic-linolenic acid triglycerides | 1.7% |
| palmitic-oleic-linoleic acid triglycerides | 13.5% |
| sodium hyaluronate | 0.1% |
| preserving agents | 0.1% |
| vitamin | 0.5% |
| UV-screening agent | 0.7% |

A composition comprising 1% of dye according to the invention and 99% of the above base is thus prepared.

The L, a and b measurements were performed on a Leneta brand contrast card of reference Form 1A Penopac, for a composition spread to a thickness of 50 microns.

The reflection measurements were performed using a Minolta 3700D spectrocolorimeter.

The initial trichromatic coordinates of the composition before irradiation (L₀, a₀ and b₀) were thus determined.

The compositions were then subjected to a UV irradiation (flux of 2 mW/cm² of a UVA radiation) for two minutes and then, immediately after stopping the irradiation, the trichromatic coordinates after irradiation (L, a and b) were determined. Less than 5 seconds elapsed between the stopping of the irradiation and the determination of the new coordinates.

The ΔE was then calculated in the following manner:
ΔE=[(L−L₀)²+(a−a₀)²+(b−b₀)²]^{1/2}

The following results are obtained:

| Dye | ΔE |
|---|---|
| 3,3-Di(4-methoxyphenyl)-6-morpholino-3H-naphtho- | 51 |
| [2,1-b]pyran | |
| 3-Phenyl-3-(4-morpholinophenyl)-6-morpholino-3H- | 53 |
| naphtho[2,1-b]pyran | |
| 3-Phenyl-3-(4-piperidinophenyl)-6-morpholino-3H- | 49 |
| naphtho[2,1-b]pyran | |

A ΔE measurement of greater than about 30 means that the dye gives a bright color; it is found that this is particularly the case with the dyes according to the invention tested herein.

### EXAMPLE 6

In order to demonstrate the solvatochromism of the dyes according to the invention, i.e. the influence of the nature of the oil on the color expression of the photochromic dyes, without exposure to UV, several simple mixtures were prepared comprising 0.05% by weight of 3-phenyl-3-(4-piperidinophenyl)-6-morpholino-3H-naphtho[2,1-b]pyran (Reversacol Ruby from James Robinson) in various oils.

The trichromatic coordinates (Lₕ, aₕ and bₕ) of the oil alone and of the oil+dye mixture (Lₘ, aₘ and bₘ) were thus determined, and the ΔE′ was calculated in the following manner:
ΔE′=[Lₕ−Lₘ)²+(aₕ−aₘ)²+(bₕ−bₘ)²]^{1/2}.

To do this, a Minolta 3700D spectrocolorimeter in transmission mode was used, with a plastic cuvette (“cuvette walls+oil+optional dye” optical path length=1 cm).

The following results are obtained:

| | δₐ of the oil alone | ΔE′ of the oil + |
|---|---|---|
| Oil | (J/cm³)^{1/2} | dye mixture |
| Castor oil | 9.09 | 33.6278 |
| Octyldodecanol | 7.69 | 37.6049 |
| Diisostearyl malate | 7.19 | 12.9041 |
| Capric/caprylic acid | 6.69 | 12.4093 |
| triglyceride | | |
| Triisononanoin | 6.54 | 12.8122 |
| Tridecyl | 5.35 | 14.2178 |
| trimellitate | | |
| Isononyl | 4.87 | 7.7386 |
| isononanoate | | |
| Hydrogenated | 0 | 6.6920 |
| polyisobutene | | |
| Isododecane | 0 | 4.2421 |

The solvatochromic effect is thus observed, i.e. the color change due to the oil, is proportionately greater the more polar the oil.

### EXAMPLE 7

The mixtures prepared in Example 6 were irradiated with UV (flux of 2 mW/cm² of UVA) for 2 minutes.

The trichromatic coordinates of the mixture after irradiation (L_{m′}, a_{m′} and b_{m′}) were again determined, as in Example 2, and the ΔE was calculated in the following manner:
ΔE=[(L_{m′}−Lₘ)²+(a_{m′}−aₘ)²+(b_{m′}−bₘ)²]^{1/2}.

Less than 5 seconds elapsed between stopping the irradiation and determining the new coordinates.

The following results are obtained:

| Oil | ΔE |
|---|---|
| Castor oil | 19.02 |
| Octyldodecanol | 20.88 |
| Diisostearyl malate | 38.79 |
| Capric/caprylic acid triglyceride | 23.70 |
| Triisononanoin | 21.62 |
| Tridecyl trimellitate | 13.74 |
| Isononyl isononanoate | 25.47 |
| Hydrogenated polyisobutene | 17.69 |
| Isododecane | 19.17 |

It is thus clearly found that the photochromic effect does indeed exist for the dye dissolved in various oils.

### EXAMPLE 8

15% by weight of polyethylene wax (apolar wax) was added to the mixtures prepared in Example 6 (oils+0.05% by weight of Reversacol Ruby dye), and the trichromatic coordinates L, a and b were then measured, before and after irradiation with UV for 2 minutes (flux of 2 mW/cm² of UVA) on thin films of composition, 50 microns thick, poured onto a contrast card.

To do this, a Minolta 3700D spectrocolorimeter in reflection mode was used; less than 5 seconds elapsed between stopping the irradiation and determining the new coordinates.

As previously, the ΔE was calculated and the following results are obtained:

| Nature of the oil in the composition | |
|---|---|
| (15% wax + 0.05% dye) | ΔE |
| Castor oil | 26.75 |
| Octyldodecanol | 31.09 |
| Diisostearyl malate | 43.12 |
| Triisononanoin | 33.70 |
| Tridecyl trimellitate | 31.98 |
| Isononyl isononanoate | 27.74 |
| Hydrogenated polyisobutene | 18.36 |
| Isododecane | 27.21 |

It is thus found that the photochromic effect still exists in a structured system, i.e. in the presence of a wax.

### EXAMPLE 9

An apolar lip gloss was prepared, comprising (% by weight):

| | |
|---|---|
| Reversacol Ruby | 0.05% |
| polybutylene (δₐ = 0) | 99.95% |

A gel with a very pale salmon initial color is obtained, which becomes deep salmon after exposure to sunlight for a few seconds. The photochromic effect is all the more pronounced the less colored and transparent the starting composition. The speed of the UV-excitation effect and likewise of the relaxation is noteworthy.

### EXAMPLE 10

A polar lip gloss was prepared, comprising (% by weight):

| | |
|---|---|
| Reversacol Ruby | 0.2% |
| 2-octyldodecanol | 10% |
| fumed silica | 6% |
| castor oil | qs 100% |
| δₐ of the oily phase > 7 (J/cm³)^{1/2} | |

A semi-thick gel with a dark garnet initial color is obtained. The solvatochromic effect is very visible.

### EXAMPLE 11

A lipstick was prepared, comprising (% by weight):

| | |
|---|---|
| Reversacol Ruby | 0.2% |
| polyethylene wax | 15% |
| hydrogenated polyisobutene (δₐ = 0) | qs 100% |

A stick with a very pale orange-white initial color is obtained, which becomes a very strong dark orange after exposure to sunlight for a few seconds.

The return to the initial color is also rapid.

### EXAMPLE 12

A lipstick was prepared, comprising (% by weight):

| | |
|---|---|
| Reversacol Ruby | 0.05% |
| 2-octyldodecanol | 10% |
| fluid lanolin oil | 10% |
| polyethylene wax | 15% |
| castor oil | qs 100% |

A stick with a pale pink initial color is obtained, which becomes a very strong dark orange after exposure to sunlight for a few seconds.

A very large photochromic effect is observed, with a high quality of return to the unexcited state in terms of speed and recovery of the absence of color (the return takes place, like the excitation, within a few seconds).

The above written description of the invention provides a manner and process of making and using it such that any person skilled in this art is enabled to make and use the same, this enablement being provided in particular for the subject matter of the appended claims, which make up a part of the original description.

As used above, the phrase “selected from the group consisting of” includes mixtures of the mentioned materials.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in a cosmetically acceptable medium, at least one oily phase and at least one photochromic organic dye of formula (I) or (II): 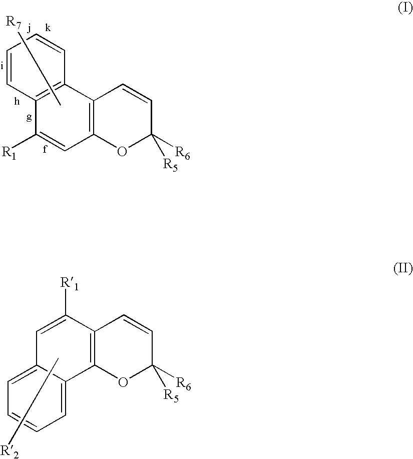
in which:
R1 represents:
(i) a hydrogen atom;
(ii) a linear, branched or cyclic, saturated or unsaturated hydrocarbon-based group containing from 1 to 30 carbon atoms, optionally comprising 1 to 5 hetero atoms selected from the group consisting of N, O, S, Si and P, and/or optionally halogenated or perhalogenated;
(iii) a hydrocarbon-based ring formed with one of the bonds “f” or “gh” and the radical R7; or
(iv) a group selected from the group consisting of —COOR4, —C(O)NR2R3, —NR2R3, —OR4 and —SR4, in which:
R2 and R3 either represent, independently of each other, a linear, branched or cyclic, saturated or unsaturated hydrocarbon-based group containing 1 to 20 carbon atoms, optionally comprising 1 to 5 hetero atoms selected from the group consisting of N, O, S, Si and P,
or, taken together with the nitrogen atom to which they are attached, form a saturated or unsaturated hydrocarbon-based heterocycle containing 3-10 carbon atoms and optionally 1-5 other hetero atoms selected from the group consisting of N, O, S, Si and P, the ring optionally being substituted with at least one linear, branched or cyclic, saturated or unsaturated hydrocarbon-based radical containing 1 to 20 carbon atoms, optionally comprising 1 to 5 hetero atoms selected from the group consisting of N, O, S, Si and P;
R4 represents a linear, branched or cyclic, saturated or unsaturated hydrocarbon-based group containing 1 to 20 carbon atoms, which is optionally halogenated or perhalogenated and/or optionally comprising 1 to 5 hetero atoms selected from the group consisting of N, O, S, Si and P;
R5 and R6 represent, independently of each other, a group selected from the group consisting of:
(i) the saturated cyclic aminoaryl groups of formula (IIa) or (IIb): 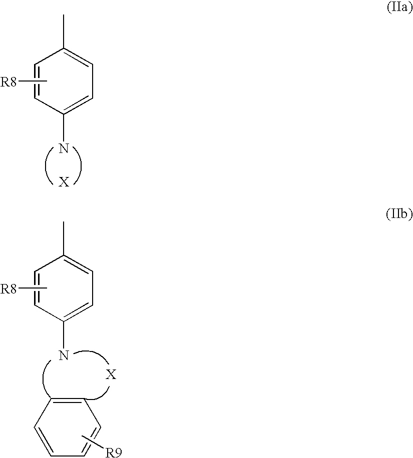
in which the ring comprising N and X is a saturated ring containing in total 3 to 30 atoms, including nitrogen, the remainder being carbon atoms and/or hetero atoms selected from the group consisting of O, S, Si and P and/or groups selected from the group consisting of —NH and —NR with R representing a linear, branched or cyclic, saturated or unsaturated hydrocarbon-based radical containing 1 to 20 carbon atoms, optionally comprising 1 to 5 hetero atoms selected from the group consisting of N, O, S, Si and P;
(ii) the indolinoaryl groups of formula (III): 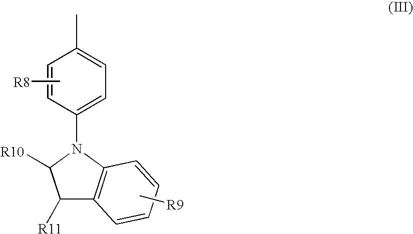
in which R10 and R11 represent, independently of each other, a group selected from the group consisting of (i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated; (ii) halogen atoms; (iii) —CN (nitrile), —COOH (carboxylate) or —NO₂ (nitro) groups; (iv) a hydrogen atom; (v) a group selected from the group consisting of —C(O)NR2R3, —NR2R3, —OR4 and —SR4 with R2, R3 and R4 having the meanings given above; (vi) the radicals R10 and R11 together possibly forming a saturated or unsaturated hydrocarbon-based ring containing in total 5 to 8 atoms (including the atoms of the indoline ring), the atoms being selected from the group consisting of C, O, S and/or NR with R representing H or a linear, branched or cyclic, saturated or unsaturated hydrocarbon-based radical containing 1 to 20 carbon atoms, optionally comprising 1 to 5 hetero atoms selected from the group consisting of N, O, S, Si and P,
(iii) the groups of formula (IV): 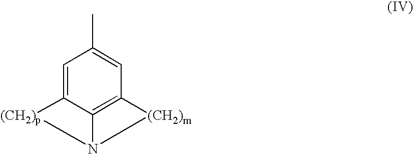
in which m and p are, independently of each other, integers ranging from 2 to 5;
(iv) the unsaturated cyclic aminoaryl groups of formula (Va), (Vb) or (Vc): 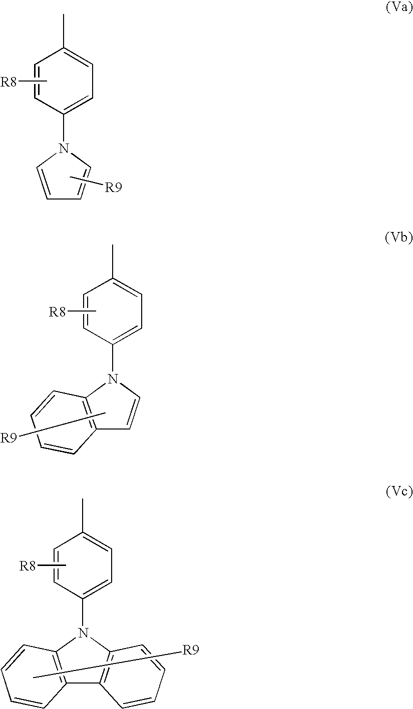
in which R8 and R9 represent, independently of each other, a group selected from the group consisting of (i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated; (ii) halogen atoms; (iii) —CN (nitrile), —COOH (carboxylate) or —NO₂ (nitro) groups; (iv) a hydrogen atom; (v) a group selected from the group consisting of —C(O)NR2R3, —NR2R3, —OR4 and —SR4 with R2, R3 and R4 having the meanings given above;
(v) a linear, branched or cyclic, saturated or unsaturated hydrocarbon-based group containing 1 to 30 carbon atoms, optionally comprising 1 to 5 hetero atoms selected from the group consisting of N, O, S, Si and P;
R7 represents a group selected from the group consisting of:
(i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated;
(ii) halogen atoms;
(iii) —CN (nitrile), —COOH (carboxylate), —NO₂ (nitro), —N═N— (azo), ═NH (imino) or —CONH₂ (amide) groups;
(iv) a hydrogen atom;
(v) a group selected from the group consisting of —C(O)NR2R3, —NR2R3, —OR4 and —SR4 with R2, R3 and R4 having the meanings given above;
(vi) the radical R7 also possibly forming, with one of the bonds “i”, “j”, “k” or “g,h” taken with the radical R1, or “f” taken with the radical R1, a saturated hydrocarbon-based ring containing in total 3 to 8 carbon atoms, optionally comprising 1 to 5 hetero atoms selected from the group consisting of N, O, S, Si and P;
R′1 represents a group selected from the group consisting of:
(i) a hydrogen atom;
(ii) a linear, branched or cyclic, saturated or unsaturated hydrocarbon-based group containing 1 to 30 carbon atoms, optionally comprising 1 to 5 hetero atoms selected from the group consisting of N, O, S, Si and P, and/or optionally halogenated or perhalogenated;
(iii) a group selected from the group consisting of —C(O)NR2R3, —NR2R3, —OR4 and —SR4, with R2, R3 and R4 having the meanings given above;
R′2 represents a group selected from the group consisting of:
(i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated;
(ii) halogen atoms;
(iii) —CN (nitrile), —COOH (carboxylate), —NO₂ (nitro), —N═N— (azo), ═NH (imino) or —CONH₂ (amide) groups;
(iv) a hydrogen atom;
(v) a group selected from the group consisting of —C(O)NR2R3, —NR2R3, —OR4 and —SR4, with R2, R3 and R4 having the meanings given above,
the organic dye being soluble in the oily phase of the composition.

2. The composition according to claim 1, in which the photochromic organic dye corresponds to one of the formulae (Ia) and (IIa) below: 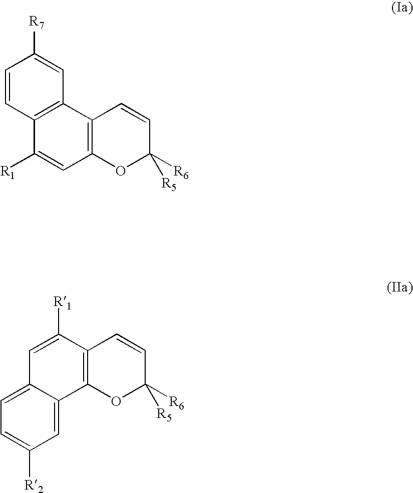
in which R1, R5, R6, R7, R′1 and R′2 are defined as in claim 1.

3. The composition according to claim 1, in which R1 represents a hydrogen atom; a hydrocarbon-based ring with one of the bonds “f” or “gh” and the radical R7; or a group selected from the group consisting of —COOR4, —NR2R3, —OR4 and —SR4, in which:
R2 and R3 either represent, independently of each other, a linear, branched or cyclic, saturated or unsaturated hydrocarbon-based group containing 1 to 20 carbon atoms, optionally comprising 1 to 5 hetero atoms selected from the group consisting of N, O, S, Si and P,
or, taken together with the nitrogen atom to which they are attached, form a saturated or unsaturated hydrocarbon-based heterocycle containing 3-10 carbon atoms and optionally 1-5 other hetero atoms selected from the group consisting of N, O, S, Si and P, the ring optionally being substituted with at least one linear, branched or cyclic, saturated or unsaturated hydrocarbon-based radical containing 1 to 20 carbon atoms, optionally comprising 1 to 5 hetero atoms selected from the group consisting of N, O, S, Si and P;
R4 represents a linear, branched or cyclic, saturated or unsaturated hydrocarbon-based group containing 1 to 20 carbon atoms, optionally halogenated or perhalogenated, and/or optionally comprising 1 to 5 hetero atoms selected from the group consisting of N, O, S, Si and P.

4. The composition according to claim 1, in which R5 and R6 represent, independently of each other, a group selected from the group consisting of:
the saturated cyclic aminoaryl groups of formula (IIa) or (IIb): 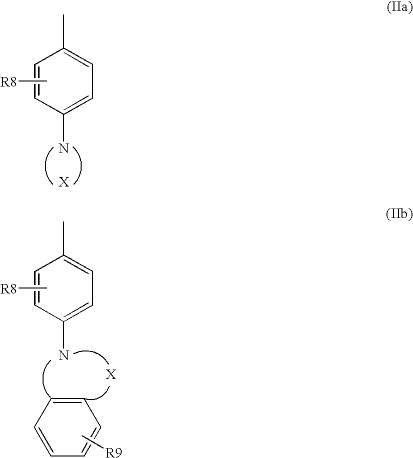
in which the ring comprising N and X is a saturated ring which contains in total 3 to 30 atoms, including nitrogen, the remainder being carbon atoms and/or hetero atoms selected from the group consisting of O, S, Si and P and/or groups selected from the group consisting of —NH and —NR with R representing a linear, branched or cyclic, saturated or unsaturated hydrocarbon-based radical containing 1 to 20 carbon atoms, optionally comprising 1 to 5 hetero atoms selected from the group consisting of N, O, S, Si and P;
a linear, branched or cyclic, saturated or unsaturated hydrocarbon-based group containing 1 to 30, preferably 2-18 carbon atoms, optionally comprising 1 to 5 hetero atoms selected from the group consisting of N, O, S, Si and P; with R2, R3 and R4 having the meanings given in claim 1.

5. The composition according to claim 1, in which R7 represents a group selected from the group consisting of:
(i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated;
(ii) halogen atoms, especially F, Br and/or Cl;
(iii) —CN (nitrile), —COOH (carboxylate), —NO₂ (nitro), —N═N— (azo), ═NH (imino) or —CONH₂ (amide) groups;
(iv) a hydrogen atom;
(v) a group selected from the group consisting of —NR2R3, —OR4 and —SR4, with R2, R3 and R4 having the meanings given in claim 1;
(vi) the radical R7 also possibly forming, with one of the bonds “i”, “j”, “k” or “g,h” taken with the radical R1, or “f” taken with the radical R1, a saturated hydrocarbon-based ring containing in total 3 to 8 carbon atoms, optionally comprising 1 to 5 hetero atoms selected from the group consisting of N, O, S, Si and P.

6. The composition according to claim 1, in which R′1 represents hydrogen or a linear, branched or cyclic, saturated or unsaturated hydrocarbon-based group containing 1 to 30 carbon atoms, optionally comprising 1 to 5 hetero atoms selected from the group consisting of N, O, S, Si and P, and/or optionally halogenated or perhalogenated.

7. The composition according to claim 1, in which R12 represents hydrogen or a group selected from the group consisting of —NO₂, —NR2R3 and —C(O)NR2R3, in which R2 and R3 either represent, independently of each other, a linear, branched or cyclic, saturated or unsaturated hydrocarbon-based group containing 1 to 20 carbon atoms, optionally comprising 1 to 5 hetero atoms selected from the group consisting of N, O, S, Si and P; or, taken together with the nitrogen atom to which they are attached, form a saturated or unsaturated hydrocarbon-based heterocycle containing 3-10 carbon atoms and optionally 1-5 other hetero atoms selected from the group consisting of N, O, S, Si and P, the ring optionally being substituted with at least one linear, branched or cyclic, saturated or unsaturated hydrocarbon-based radical containing 1 to 20 carbon atoms, optionally comprising 1 to 5 hetero atoms selected from the group consisting of N, O, S, Si and P.

8. The composition according to claim 1, in which the organic dye is of formula (I) or (Ia) for which:
R1 represents hydrogen; or a group —COOR with R being a saturated hydrocarbon-based radical containing 1 to 12 carbon atoms; or a group 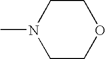
and/or
R5 and R6 represent, independently of each other, either (i) a group of formula (IIa): 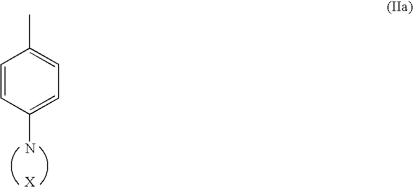
in which the ring comprising N and X is a saturated ring selected from the group consisting of groups of formulae: 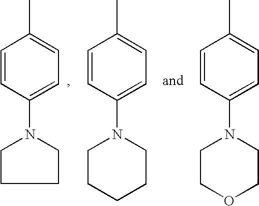
or (ii) a linear, branched or cyclic, saturated or unsaturated hydrocarbon-based group containing 5 to 14 carbon atoms, optionally comprising 1 or 2 hetero atoms selected from the group consisting of 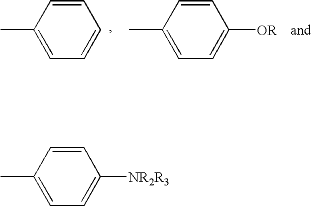
in which R is a saturated hydrocarbon-based radical containing 1 to 12 carbon atoms; and R2 and R3 are, independently of each other, a linear, branched or cyclic, saturated or unsaturated hydrocarbon-based group containing 1 to 20 carbon atoms, optionally comprising 1 to 5 hetero atoms selected from the group consisting of N, O, S, Si and P; and/or
R7 represents a hydrogen atom or a group —NR2R3, with R2 and R3 representing, independently of each other, a linear or branched, saturated hydrocarbon-based group containing 1 to 12 carbon atoms.

9. The composition according to claim 1, in which the organic dye is of formula (II) for which:
R′1 represents hydrogen or a group —COOR with R being a saturated hydrocarbon-based radical containing 1 to 12 carbon atoms; and/or
R5 and R6 represent, independently of each other, either (i) a group of formula (IIa): 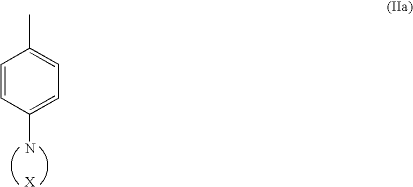
in which the ring comprising N and X is a saturated ring containing in total 4 to 7 atoms including nitrogen;
or (ii) a linear, branched or cyclic, saturated or unsaturated hydrocarbon-based group containing 5 to 14 carbon atoms, optionally comprising 1 or 2 hetero atoms selected from the group consisting of N, O and S; and/or
R′2 represents hydrogen or a group —NR′R″, with R′ and R″, which may be identical or different, representing a linear or branched, saturated hydrocarbon-based group containing 1 to 12 carbon atoms.

10. The composition according to claim 1, in which the organic dye is selected from the group consisting of:
3,3-di(4-methoxyphenyl)-6-morpholino-3H-naphtho-[2,1-b]pyran of formula: 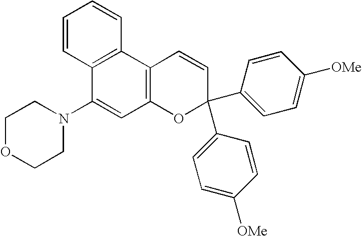
3-phenyl-3-(4-morpholinophenyl)-6-morpholino-3H-naphtho[2,1-b]pyran of formula: 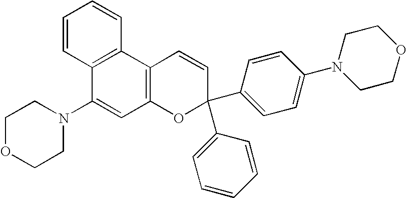
3-phenyl-3-(4-piperidinophenyl)-6-morpholino-3H-naphtho[2,1-b]pyran of formula: 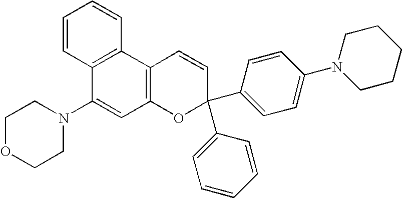
3-phenyl-3-(4-piperidinophenyl)-6-methoxycarbonyl 9-N-dimethyl-3H-naphtho[2,1-b]pyran of formula: 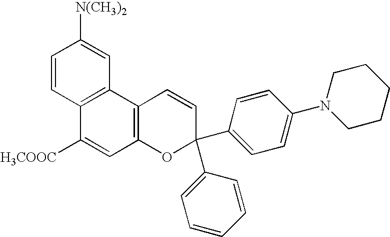
2-phenyl-2-(4-piperidinophenyl)-5-methoxycarbonyl-9-N-dimethyl-2H-naphtho[1,2-b]pyran of formula: 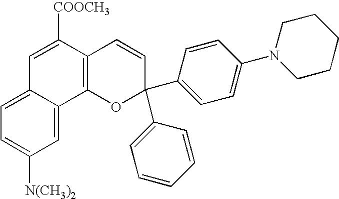
and mixtures thereof.

11. The composition according to claim 1, in which the organic dye is included, alone or as a mixture, in an amount of from 0.001% to 20% by weight relative to the total weight of the cosmetic composition.

12. The composition according to claim 1, in which the oily phase is polar and has a mean solubility parameter δₐ according to the Hansen solubility space, at 25° C., of greater than or equal to 5.0 (J/cm³)^{1/2}.

13. The composition according to claim 1, in which the oily phase comprises 5% to 100% by weight, relative to the total weight of the oily phase, of one or more polar oils with a mean solubility parameter δₐ according to the Hansen solubility space, at 25° C., of greater than or equal to 5.0 (J/cm³)^{1/2}.

14. The composition according to claim 1, in which the oily phase is apolar and has a mean solubility parameter δₐ according to the Hansen solubility space, at 25° C., of less than 5.0.

15. The composition according to claim 1, in which the oily phase comprises 5% to 100% by weight, relative to the total weight of the oily phase, of one or more apolar oils with a mean solubility parameter δₐ according to the Hansen solubility space, at 25° C., of less than 5.0 (J/cm³)^{1/2}.

16. The composition according to claim 1, in which the oily phase comprises at least one oil selected from the group consisting of:
animal or plant oils formed from fatty acid esters of polyols; fish oils or glyceryl tricaprocaprylate, or plant or animal oils of formula R₁COOR₂ in which R₁ represents a higher fatty acid residue containing from 7 to 19 carbon atoms and R₂ represents a branched hydrocarbon-based chain containing from 3 to 20 carbon atoms; liquid paraffin, liquid petroleum jelly, beauty-leaf oil, macadamia oil, rapeseed oil, coconut oil, groundnut oil, palm oil, castor oil, jojoba oil, olive oil or cereal germ oil; shea butter oil;
perhydrosqualene;
synthetic esters and ethers; polyol esters; tridecyl trimellitate;
fatty alcohols containing from 12 to 26 carbon atoms;
linear or branched hydrocarbons of mineral or synthetic origin; isoparaffins;
glycerides:
and mixtures thereof.

17. The composition according to claim 1, in which the oily phase comprises at least one oil selected from the group consisting of octyldodecanol, hexyldecanol, octyldecanol, oleyl alcohol, castor oil, diisostearyl malate, glyceryl triheptanoate, glyceryl trioctanoate, capric/caprylic acid triglyceride, triisononanoin, tridecyl trimellitate, C6-C40 aliphatic hydrocarbons, petroleum jelly, hydrogenated or non-hydrogenated polydecenes, hydrogenated polyisobutene, squalane, polybutylenes and isononyl isononanoate; fluoro oils, and mixtures thereof.

18. The composition according to claim 1, in which the oily phase comprises at least one phenylsilicone oil of formula (VI), or a mixture of such oils: 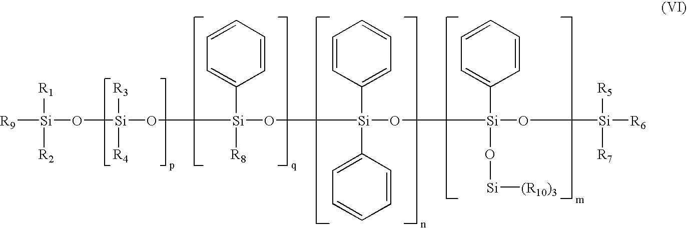
in which
—R1 to R10, independently of each other, are saturated or unsaturated, linear, cyclic or branched C₁-C₃₀ hydrocarbon-based radicals,
m, n, p and q are, independently of each other, integers between 0 and 900, with the proviso that the sum m+n+q is other than 0.

19. The composition according to claim 18, in which, in the phenylsilicone oil of formula (VI), the sum m+n+q is between 1 and 100 and/or the sum m+n+p+q is between 1 and 900.

20. The composition according to claim 18, comprising a phenylsilicone oil of formula (VII): 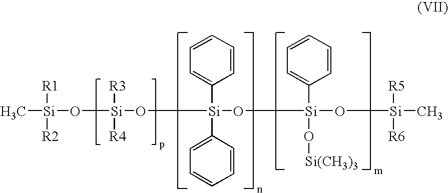
in which:
R1 to R6, independently of each other, are saturated or unsaturated, linear, cyclic or branched C₁-C₃₀ hydrocarbon-based radicals,
m, n and p are, independently of each other, integers between 0 and 100, with the proviso that the sum n+m is between 1 and 100.

21. The composition according to claim 18, in which, in formulae (VI), R1 to R6, independently of each other, represent a saturated linear or branched C₁-C₃₀ hydrocarbon-based radical.

22. The composition according to claim 18, in which, in formulae (VI), R1 to R6 are identical and are a methyl radical.

23. The composition according to claim 18, in which the phenylsilicone oil is selected from the group consisting of phenyl trimethicones, phenyl dimethicones, phenyl trimethylsiloxy diphenylsiloxanes, diphenyl dimethicones, diphenyl methyldiphenyl trisiloxanes, and mixtures thereof.

24. The composition according to claim 18, in which the phenylsilicone oil is present in an amount of between 5-90% by weight, relative to the total weight of the composition.

25. The composition according to claim 18, in which the oily phase of the composition comprises one or more other additional cosmetically acceptable, polar or apolar, volatile or non-volatile, silicone-based or hydrocarbon-based oils, which is present in an amount of between 0.1-50% by weight, relative to the total weight of the cosmetic composition.

26. The composition according to claim 1, in which the oily phase is included in an amount of from 10% to 90% by weight relative to the total weight of the cosmetic composition.

27. The composition according to claim 1, further comprising other fatty substances selected from the group consisting of waxes, gums and/or pasty fatty substances of animal, plant, mineral or synthetic origin, and also mixtures thereof; and/or a particulate phase, which may comprise pigments, nacres, fillers, dyes and/or other photochromic compounds; and/or UV-screening agents; and/or an aqueous phase; and/or a surfactant and/or a thickener and/or a film-forming polymer.

28. The composition according to claim 1, having a ΔE value of greater than or equal to 5.

29. The composition according to claim 1, which is in the form of a care and/or makeup product for body or facial skin, for the lips and for the hair, an antisun product or self-tanning product, or a hair product.

30. The composition according to claim 1, which is in the form of a lipstick, a foundation, a makeup rouge, an eyeshadow, a free or compact powder, a tinted cream, a body makeup product, a skin-coloring product, an eyeliner or a mascara.

31. A process for treating a support selected from the group consisting of mucous membranes, semi-mucous membranes, the skin and/or the integuments, comprising applying the composition of claim 1 to the support.
